(19) United States
(12) Patent Application Publication
(10) Pub. No.: US 2025/0375666 A1
(43) Pub. Date: Dec. 11, 2025

(54) SYSTEMS, DEVICES, AND METHODS FOR ATHLETIC AND/OR SKILLS ASSESSMENT, DEVELOPMENT, AND/OR PERFORMANCE

(71) Applicant: BALLOGY, INC., Austin, TX (US)

(72) Inventors: Todd Young, Austin, TX (US); Joyce Ekworomadu, Dallas, TX (US); Jordan Green, Austin, TX (US); Syed Saad Hussain, Lahore (PK)

(21) Appl. No.: 19/073,001

(22) Filed: Mar. 6, 2025

Related U.S. Application Data
(60) Provisional application No. 63/562,679, filed on Mar. 7, 2024.

(51) Int. Cl. A63B 24/00; A63B 69/00

(52) U.S. Cl. CPC A63B 24/0075; A63B 69/0071; A63B 2024/0081; A63B 2243/0037

(57) ABSTRACT

The present disclosure relates generally to devices and methods for athletic and/or skills assessment, development, and/or performance.

The present patent application claims priority from U.S. provisional patent application Ser. No. 63/562,679, entitled “SYSTEMS, DEVICES, AND METHODS FOR ATHLETIC AND/OR OR SKILLS ASSESSMENT, DEVELOPMENT, AND/OR PERFORMANCE,” filed Mar. 7, 2024, the contents of which are incorporated herein by reference in their entirety and for all purposes.

### BACKGROUND

### Field

The present disclosure relates generally to devices and methods for athletic and/or skills assessment, development, and/or performance.

### Information

In athletic competition and/or other skilled endeavors, coaching and/or recruiting are increasingly moving from paper to digital. In circumstances, coaches, for example, may distribute hardcopies of drills and plays, take paper notes, score drills on paper sheets, etc. To help organize such materials, coaches and/or recruiters may, in some circumstances, use multiple third party systems to compile data, translate documents into digital form, communicate with players, evaluate and/or score players in various drills or activities, predict performance, etc. In some circumstances, such third party systems may not be compatible with each other. Such use of non-compatible third-party systems may lead to a disproportionate amount of time and effort spent by coaches and/or administrators, for example, in performing administrative tasks rather than substantive coaching and/or recruiting, for example.

### BRIEF DESCRIPTION OF THE DRAWINGS

Claimed subject matter is particularly pointed out and distinctly claimed in the concluding portion of the specification. However, both as to organization and/or method of operation, together with objects, features, and/or advantages thereof, it may best be understood by reference to the following detailed description if read with the accompanying drawings in which:

FIG. 1 is a schematic block diagram depicting an embodiment of an example system including one or more server computing devices and/or one or more IoT-type devices;

FIG. 2 is a schematic block diagram depicting an embodiment of an example Internet of Things (IoT)-type device;

FIGS. 3A-3B, including associated subsections, depicts aspects of a graphical user interface related to team training and/or workouts, including automatic assignment-type curriculum Workout plans, for a sports social media application, in one or more embodiments;

FIG. 4, including associated subsections, depicts aspects of a graphical user interface related to Workout creation and/or assignment for a sports social media application, in one or more embodiments;

FIGS. 5A-5E, including associated subsections, depict aspects of a graphical user interface related to assigned Workouts and/or tasks for a sports social media application, in one or more embodiments;

FIGS. 6A-6B, including associated subsections, depicts aspects of a graphical user interface related to a skills assessment for a sports social media application, in one or more embodiments;

FIG. 7 depicts a flow diagram of an example process for automatic assignment-type curriculum Workout plans, in one or more embodiments;

FIG. 8 depicts a flow diagram of additional aspects of an example process for automatic assignment-type curriculum Workout plans, in one or more embodiments;

FIG. 9 depicts a schematic diagram illustrating an implementation of an example computing environment according to an embodiment;

FIG. 10 is a schematic diagram of a neural network formed in “layers”, according to an embodiment; and

FIG. 11 is a flow diagram of an aspect of a training operation, according to an embodiment.

Reference is made in the following detailed description to accompanying drawings, which form a part hereof, wherein like numerals may designate like parts throughout that are corresponding and/or analogous. It will be appreciated that the figures have not necessarily been drawn to scale, such as for simplicity and/or clarity of illustration. For example, dimensions of some aspects may be exaggerated relative to others. Further, it is to be understood that other embodiments may be utilized. Furthermore, structural and/or other changes may be made without departing from claimed subject matter. References throughout this specification to “claimed subject matter” refer to subject matter intended to be covered by one or more claims, or any portion thereof, and are not necessarily intended to refer to a complete claim set, to a particular combination of claim sets (e.g., method claims, apparatus claims, etc.), or to a particular claim. It should also be noted that directions and/or references, for example, such as up, down, top, bottom, and so on, may be used to facilitate discussion of drawings and are not intended to restrict application of claimed subject matter. Therefore, the following detailed description is not to be taken to limit claimed subject matter and/or equivalents. It should also be noted that “subject matter” and “claimed subject matter” can be used interchangeably herein.

### DETAILED DESCRIPTION

References throughout this specification to one implementation, an implementation, one embodiment, an embodiment, and/or the like means that a particular feature, structure, characteristic, and/or the like described in relation to a particular implementation and/or embodiment is included in at least one implementation and/or embodiment of claimed subject matter. Thus, appearances of such phrases, for example, in various places throughout this specification are not necessarily intended to refer to the same implementation and/or embodiment or to any one particular implementation and/or embodiment. Furthermore, it is to be understood that particular features, structures, characteristics, and/or the like described are capable of being combined in various ways in one or more implementations and/or embodiments and, therefore, are within intended claim scope. In general, of course, as has always been the case for the specification of a patent application, these and other issues have a potential to vary in a particular context of usage. In other words, throughout the patent application, particular context of description and/or usage provides helpful guidance regarding reasonable inferences to be drawn; however, likewise, “in this context” in general without further qualification refers to the context of the present patent application.

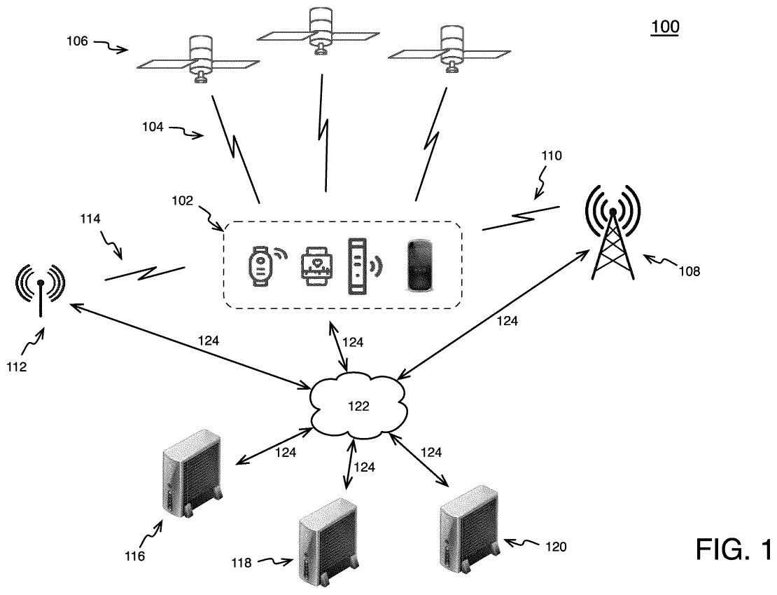

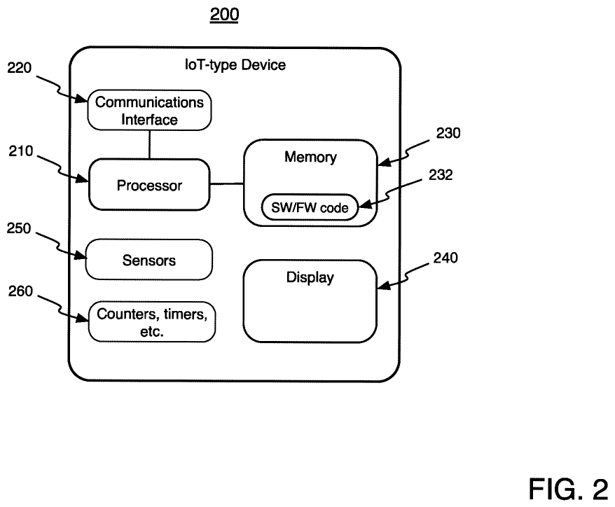

Even though a number of embodiments and/or implementations are described herein, it should be noted, however, that subject matter is not limited in scope to the particular example embodiments and/or implementations provided. Also, example embodiments and/or implementations may utilize, at least in part, any of a wide range of computing and/or communication devices, systems, components, technologies, networks, etc. The discussion below, including aspects related to FIGS. 1 and 2, for example, describes various aspects of an example infrastructure that may facilitate and/or support, at least in part, one or more example embodiments and/or implementations discussed herein.

The “World Wide Web” or simply the “Web,” such as provided by the Internet, for example, is growing rapidly, at least in part, from the large amount of content being added seemingly on a daily basis. A wide variety of content in the form of stored signals, such as, for example, text files, images, audio files, video files, web pages, measurements of physical phenomena, and/or the like may be continually acquired, identified, located, retrieved, collected, stored, communicated, etc. Increasingly, content is being acquired, collected, communicated, etc. by a number of electronic devices, such as, for example, embedded computing devices leveraging existing Internet and/or like infrastructure as part of a so-called “Internet of Things” (IoT), such as via a variety of protocols, domains, and/or applications. IoT may typically comprise a system of interconnected and/or internetworked physical computing devices capable of being identified, such as uniquely via an assigned Internet Protocol (IP) address, for example. Devices, such as IoT-type devices, for example, may include computing resources embedded into hardware so as to facilitate and/or support a device's ability to acquire, collect, process and/or transmit content over one or more communications networks. In this context, “IoT-type devices” and/or the like refer to one or more electronic and/or computing devices capable of leveraging existing Internet and/or like infrastructure as part of the IoT, such as via a variety of applicable protocols, domains, applications, etc. In particular implementations, IoT-type devices, for example, may comprise a wide variety of embedded devices, such as, for example, automobile sensors, biochip transponders, heart monitoring implants, thermostats, kitchen appliances, locks or like fastening devices, solar panel arrays, home gateways, controllers, etc. and even mobile devices, desktop computers, laptop computers, and/or the like. Although embodiments described herein may refer to IoT-type devices, subject matter is not limited in scope in these respects. For example, although IoT-type devices may be described, such as for ease of discussion, it should be noted that subject matter is intended to include use of any of a wide range of electronic device types, including a wide range of computing and/or communications device types.

“Electronic content,” “digital content,” “content,” and/or the like as the terms are used herein should be interpreted broadly and refers to signals, such signal packets, for example, and/or states, such as physical states on a memory device, for example, but otherwise are employed in a manner irrespective of format, such as any expression, representation, realization, and/or communication, for example. Content may comprise, for example, any information, knowledge, and/or experience, such as, again, in the form of signals and/or states, physical or otherwise. In this context, “electronic” or “on-line” content refers to content in a form that although not necessarily capable of being perceived by a human, (e.g., via human senses, etc.) may nonetheless be transformed into a form capable of being so perceived, such as visually, haptically, and/or audibly, for example. Non-limiting examples may include text, audio, images, video, security parameters, combinations, or the like. Thus, content may be stored and/or transmitted electronically, such as before or after being perceived by human senses. In general, it may be understood that electronic content may be intended to be referenced in a particular discussion, although in the particular context, the term “content” may be employed for ease of discussion. Specific examples of content may include, for example, computer code, data, parameters, metadata, message, text, audio file, video file, data file, web page, or the like. Claimed subject matter is not intended to be limited to these particular examples, of course.

FIG. 1 is a schematic diagram illustrating features associated with an implementation of an example operating environment 100 capable of facilitating and/or supporting one or more operations and/or techniques for automatic assignment-type curriculum Workout plans, skills assessment, etc., discussed more fully below, for example, for distributed and/or remote computing and/or communications networks, illustrated generally herein at 102. As was indicated, one or more operations and/or techniques may, for example, be implemented, at least in part, in connection with one or more IoT-type devices, though subject matter is not so limited. Briefly, IoT is typically a system of interconnected and/or internetworked physical devices in which computing may be embedded into hardware so as to facilitate and/or support devices' abilities to acquire, collect and/or communicate content over one or more communications networks, for example, at times, without human participation and/or interaction. As mentioned, IoT-type devices may include a wide variety of stationary and/or mobile devices, such as, for example, automobile sensors, biochip transponders, heart monitoring implants, kitchen appliances, locks or like fastening devices, solar panel arrays, home gateways, smart gauges, smart telephones, cellular telephones, security cameras, wearable devices, thermostats, Global Positioning System (GPS) transceivers, personal digital assistants (PDAs), virtual assistants, laptop computers, personal entertainment systems, tablet or other personal computers (PCs), personal audio and/or video devices, personal navigation devices, mobile devices, and/or the like.

It should be appreciated that operating environment 100 is described herein as a non-limiting example that may be implemented, in whole or in part, in a context of various wired and/or wireless communications networks and/or any suitable portion and/or combination of such networks. For example, these or like networks may include one or more public networks (e.g., the Internet, the World Wide Web), private networks (e.g., intranets), wireless wide area networks (WWAN), wireless local area networks (WLAN, etc.), wireless personal area networks (WPAN), telephone networks, cable television networks, Internet access networks, fiber-optic communication networks, waveguide communication networks and/or the like. It should also be noted that claimed subject matter is not limited to a particular network and/or operating environment. Thus, for a particular implementation, one or more operations and/or techniques for automatic assignment-type curriculum workout plans for distributed computing and/or communications networks may be performed, at least in part, in an indoor environment and/or an outdoor environment, or any combination thereof.

Thus, as illustrated, in a particular implementation, one or more computing and/or communications devices, such as IoT-type devices 102, may, for example, receive and/or acquire satellite positioning system (SPS) signals 104 from SPS satellites 106. In some instances, SPS satellites 106 may be from a single global navigation satellite system (GNSS), such as the GPS or Galileo satellite systems, for example. In other instances, SPS satellites 106 may be from multiple GNSS such as, but not limited to, GPS, Galileo, Glonass, or Beidou (Compass) satellite systems, for example. In certain implementations, SPS satellites 1006 may be from any one several regional navigation satellite systems (RNSS) such as, for example, WAAS, EGNOS, QZSS, just to name a few examples.

At times, one or more IoT-type devices 102 may, for example, transmit wireless signals to and/or receive wireless signals from a suitable wireless communication network. In one example, one or more IoT-type devices 102 may communicate with a cellular communication network, such as by transmitting wireless signals to and/or receiving wireless signals from one or more wireless transmitters capable of transmitting and/or receiving wireless signals, such as a base station transceiver 108 over a wireless communication link 110, for example. Similarly, one or more IoT-type devices 102 may transmit wireless signals to and/or receive wireless signals from a local transceiver 112 over a wireless communication link 114, for example. Base station transceiver 108, local transceiver 112, etc. may be of the same or similar type, for example, and/or may represent different types of devices, such as access points, radio beacons, cellular base stations, femtocells, an access transceiver device, or the like, depending on an implementation. Similarly, local transceiver 112 may comprise, for example, a wireless transmitter and/or receiver capable of transmitting and/or receiving wireless signals. For example, at times, wireless transceiver 112 may be capable of transmitting and/or receiving wireless signals from one or more other terrestrial transmitters and/or receivers.

In a particular implementation, local transceiver 112 may, for example, be capable of communicating with one or more IoT-type devices 102 at a shorter range over wireless communication link 114 than at a range established via base station transceiver 108 over wireless communication link 110. For example, local transceiver 112 may be positioned in an indoor or like environment and/or may provide access to a wireless local area network (WLAN, e.g., IEEE Std. 802.11 network, etc.) and/or wireless personal area network (WPAN, e.g., Bluetooth® network, etc.). In another example implementation, local transceiver 112 may comprise a femtocell and/or picocell capable of facilitating communication via link 114 according to an applicable cellular or like wireless communication protocol. Again, it should be understood that these are merely examples of networks that may communicate with one or more IoT-type devices 102 over a wireless link, and claimed subject matter is not limited in scope in this respect. For example, in some instances, operating environment 100 may include a larger number of base station transceivers 108, local transceivers 112, networks, terrestrial transmitters and/or receivers, etc.

In an implementation, one or more IoT-type devices 102, base station transceiver 108, local transceiver 112, etc. may, for example, communicate with one or more servers, referenced herein at 116, 118, and 120, over a network 122, such as via one or more communication links 124. It should be noted that, depending on an implementation, one or more servers 116, 118, and/or 120 may be part of a centralized network, a decentralized network, or any combination thereof. Thus, even though terms like “server” or “servers' are used herein, such as for ease of discussion, it should be appreciated that these or like aspects may include and/or be part of one or more centralized and/or decentralized networks. Thus, as indicated, network 122 may comprise, for example, a centralized network, a decentralized network, or any combination thereof, and may include any number and/or combination of wired and/or wireless communication links. In a particular implementation, network 122 may comprise, for example, Internet Protocol (IP)-type infrastructure capable of facilitating or supporting communication between one or more IoT-type devices 102 and one or more servers 116, 118, 120, etc. via local transceiver 112, base station transceiver 108, directly, etc. In another implementation, network 122 may comprise, for example cellular communication network infrastructure, such as a base station controller and/or master switching center to facilitate and/or support mobile cellular communication with one or more IoT-type devices 102. Servers 116, 118 and/or 120 may comprise any suitable servers or combination thereof capable of facilitating or supporting one or more operations and/or techniques discussed herein. For example, servers 116, 118 and/or 120 may comprise one or more update servers, back-end servers, management servers, archive servers, location servers, positioning assistance servers, navigation servers, map servers, crowdsourcing servers, network-related servers, or the like.

Even though a certain number of computing platforms and/or devices are illustrated herein, any number of suitable computing platforms and/or devices may be implemented to facilitate and/or support one or more techniques and/or processes associated with operating environment 100. For example, at times, network 122 may be coupled to one or more wired and/or wireless communication networks (e.g., WLAN, etc.) so as to enhance a coverage area for communications with one or more IoT-type devices 102, one or more base station transceivers 108, local transceiver 112, servers 116, 118, 120, or the like. In some instances, network 122 may facilitate and/or support femtocell-based operative regions of coverage, for example. Again, these are merely example implementations, and claimed subject matter is not limited in this regard.

In this context, “IoT-type devices” and/or the like refer to one or more electronic and/or computing devices capable of leveraging existing Internet or like infrastructure as part of the so-called “Internet of Things” or IoT, such as via a variety of applicable protocols, domains, applications, etc. As was indicated, IoT is typically a system of interconnected and/or internetworked physical devices in which computing may be embedded into hardware so as to facilitate and/or support devices' ability to acquire, collect, and/or communicate content over one or more communications networks, for example, at times, without human participation and/or interaction. IoT-type devices 102, for example, may include a wide variety of stationary and/or mobile devices, such as, for example, automobile sensors, biochip transponders, heart monitoring implants, kitchen appliances, locks or like fastening devices, solar panel arrays, home gateways, smart gauges, smart telephones, cellular telephones, security cameras, wearable devices, thermostats, Global Positioning System (GPS) transceivers, personal digital assistants (PDAs), virtual assistants, laptop computers, personal entertainment systems, tablet personal computers (PCs), PCs, personal audio or video devices, personal navigation devices, mobile devices, stationary devices, and/or the like, to name a few non-limiting examples. Typically, in this context, a “mobile device” refers to an electronic and/or computing device that may from time to time have a position or location that changes, and/or a “stationary device” refers to an electronic and/or computing device that may have a position or location that generally does not change. In some instances, IoT-type devices, such as IoT-type devices 102, may be capable of being identified, such as uniquely, via an assigned Internet Protocol (IP) address (e.g., static, dynamic, etc.), as one particular example, and/or having an ability to communicate, such as receive and/or transmit electronic content, for example, over one or more wired and/or wireless communications networks.

It may again be noted that the example infrastructure discussed above, along with additional systems, apparatuses, processes, etc., discussed herein, may be directed, at least in part, to supporting automatic assignment-type curriculum workout plans for a sports social media application, for example. The example infrastructure discussed above, along with additional systems, apparatuses, processes, etc., discussed herein, may be further directed, at least in part, to helping coaches and/or parents, for example, to guide players, such as basketball players, in their development of skills, techniques, strategies, etc., associated with the game of basketball. As discussed more fully below, coaches, players, parents, other group owners and/or individuals may electronically communicate with other coaches, players, parents, other group owners and/or individuals and/or with one or more administrator computing devices via an application, or portions of an application, such as a sports social media application executed at least in part on their respective electronic devices. The example infrastructure described in connection with FIG. 1 and FIG. 2, for example, may provide a platform to support example functionalities described herein, including automatic assignment-type curriculum workout plans, described in more detail below. It may also be appreciated that one or more software and/or firmware agents implemented to facilitate example functionalities described herein, including automatic assignment-type curriculum workout plans, for example, may be partitioned between administrator computing devices, such as servers 120, for example, and any number of remote and/or mobile devices, such as IoT-type devices 102, for example, that may be associated with one or more coaches, players, parents, etc.

FIG. 2 is an illustration of an embodiment 200 of an example IoT-type device. Of course, subject matter is not limited in scope to the particular configurations and/or arrangements of components depicted and/or described for example devices mentioned herein. In an embodiment, an IoT-type device, such as 200, may comprise one or more processors, such as processor 210, and/or may comprise one or more communications interfaces, such as communications interface 220. In an embodiment, one or more communications interfaces, such as communications interface 220, may enable wireless and/or wired communications between an electronic device, such as an IoT-type device 200, and one or more other computing devices. In an embodiment, wireless and/or wired communications may occur substantially in accordance any of a wide range of communication protocols, such as those known and/or mentioned herein, for example, and/or developed in the future.

In a particular implementation, an IoT-type device, such as IoT-type device 200, may include a memory, such as memory 230. In a particular implementation, memory 230 may comprise a non-volatile memory, for example. Further, in a particular implementation, a memory, such as memory 230, may have stored therein executable instructions, such as for one or more operating systems, communications protocols, and/or applications, for example. A memory, such as 230, may further store particular instructions, such as software and/or firmware code 232, that may be updated via one or more example implementations and/or embodiments described herein. Further, in a particular implementation, an IoT-type device, such as IoT-type device 200, may comprise a display, such as display 240, and/or one or more sensors, such as one or more sensors 250. As utilized herein, “sensors” and/or the like refer to a device and/or component that may respond to physical stimulus, such as, for example, heat, light, sound pressure, magnetism, particular motions, etc., and/or that may generate one or more signals and/or states in response to physical stimulus. Example sensors may include, but are not limited to, one or more accelerometers, gyroscopes, thermometers, magnetometers, barometers, light sensors, proximity sensors, hear-rate monitors, perspiration sensors, hydration sensors, breath sensors, cameras, microphones, etc., and/or any combination thereof. It may be noted that IoT-type devices, such as device 200, may include one or more cameras to facilitate video recording, for example.

In particular implementations, IoT-type device 200 may include one or more timers and/or counters and/or like circuits, such as circuitry 260, for example. In an embodiment, one or more timers and/or counters and/or the like may track one or more aspects of device performance and/or operation. For example, timers, counters, and/or other like circuits may be utilized, at least in part, by IoT-type device 200 to determine measures of fitness, for example, and/or to otherwise generate feedback content related to testing results, in particular implementations.

Although FIG. 2 depicts a particular example implementation of an IoT-type device, such as IoT-type device 200, other embodiments may include other types of electronic and/or computing devices. Example types of electronic and/or computing devices may include, for example, any of a wide range of digital electronic devices, including, but not limited to, cellular telephones (e.g., smartphones), tablet devices, desktop and/or notebook computers, virtual and/or augmented reality devices, high-definition televisions, digital video players and/or recorders, game consoles, satellite television receivers, wearable devices, personal digital assistants, mobile audio and/or video playback and/or recording devices, streaming devices, or any combination of the foregoing. Of course, subject matter is not limited in scope in these respects.

In embodiments, a curriculum (e.g., automatic assignments) aspect of a sports social media application may be designed and/or implemented as a way to develop athletes, for example. A curriculum may include fundamental drills utilized in years of coaching development teams, for example, and may divide them into various (e.g., three) skill-level categories, in embodiments. In embodiments, a curriculum may be fine-tuned by a team of experienced coaches and/or professional athletes for basketball, for example, and/or may be further developed and/or fine-tuned by sports experts who may analyze fundamental basketball skills and may design progressive drills to enhance them. An opportunity to scale this concept to multiple sports has been identified and experts have been gathered from respective fields to support the building of different curricula for various individual sports, for example.

In embodiments, a sports social media application may encourage athletes to work on their skillsets at home and may take into consideration that it may be advantageous for athletes to perform additional work outside of their organized practices and/or sessions to grow and progress in their crafts. In embodiments, a curriculum may target specified skills at least in part by incorporating a diverse range of drills and/or exercises focusing on shooting, dribbling, passing, defense, and/or overall athleticism, for example. Individual drills may be carefully selected to address key aspects of player development, in embodiments.

In embodiments, a sports social media application may provide a fifty-two week Workout schedule for athletes, for example. Workouts may change throughout the year to ensure progressive improvement, in implementations. In embodiments, a pre-season period may focus on skill-building and/or conditioning. Further, for example, an in-season period may emphasize game-specific drills. Also, an off-season period may be directed to skill refinement and/or overall development, in implementations. In embodiments, pre-season and/or off-season periods may also include additional drills and/or reps within a Workout, while in-season Workouts may be directed more towards maintenance and/or respect for a coach's time in incorporating game strategies and/or concepts, for example. In embodiments, a sports social media application may offer solutions to fit a player's development in-season.

In implementations, a pre-season period may start off with drills, provided to a user via a sports social media application (e.g., a server computing device communicating with a player's remote computing device/mobile device), focusing on basic fundamentals such as defensive footwork and principles, ball-handling, shooting, finishing around the rim, etc. In embodiments, a pre-season period may begin with six drills within a Workout with an average of two or three repetitions per drill to get an athlete acclimated to player development prior to season start, for example. In embodiments, individual Workouts may be designed to take an athlete about 30-45 mins to complete. Within a pre-season, there may be ten Workouts corresponding to ten weeks of a typical pre-season period for basketball, for example.

In embodiments, in-season Workouts may comprise about two or three drills to fine-tune an athlete's ball-handling, shooting, finishing at the rim, footwork, etc., during a season, for example. Individual Workouts may be designed to last for about fifteen minutes strategically to either begin as a warm up or end practice with an exit activity, for example. Typically, a season is around twenty-two weeks in duration and therefore, in implementations, a sports social media application may provide twenty-two Workouts that may be randomized as a daily touch point for athletes, for example. In embodiments, during a season, coaches may focus on team strategies and/or concepts in a two to two-and-a-half hour practice setting, for example, and therefore a sports social media application may provide a solution to include individual player development. Of course, these are merely examples of the amounts and types of Workouts that may be selected, assigned, tracked, etc., via a sports social media application, and subject matter is not limited in scope in these respects.

In embodiments, offseason Workouts may comprise five or six drills, for example, that may start off as easing into targeted areas of refinement with a focus of improving an athlete's ball-handling, shooting, finishing at the rim, footwork, etc. Individual Workouts may be designed to last thirty to forty-five minutes and may have two phases. In embodiments, a first phase may begin with approximately three to five reps (i.e., repetitions) of each drill. In embodiments, halfway through an off-season, a level of intensity may rise in reps to challenge an athlete with a more rigorous workout, for example. In embodiments, an offseason may comprise twenty Workouts corresponding to twenty weeks for a duration of a basketball off-season in the spring and summer, for example. Again, these are merely examples of the amounts and types of Workouts that may be selected, assigned, tracked, etc., such as via a sports social media application, and subject matter is not limited in scope in these respects.

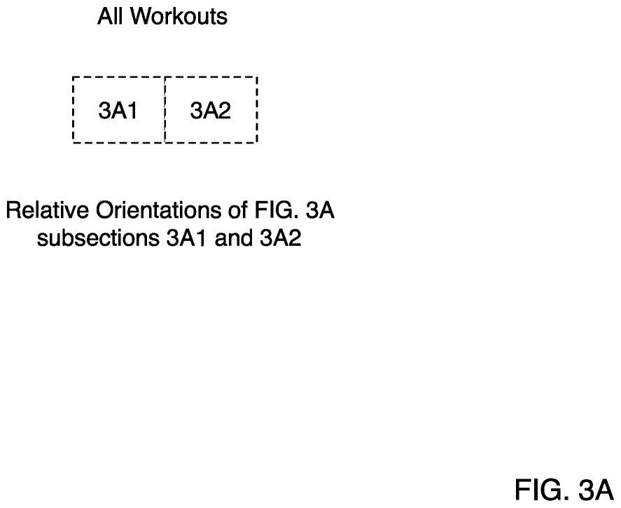

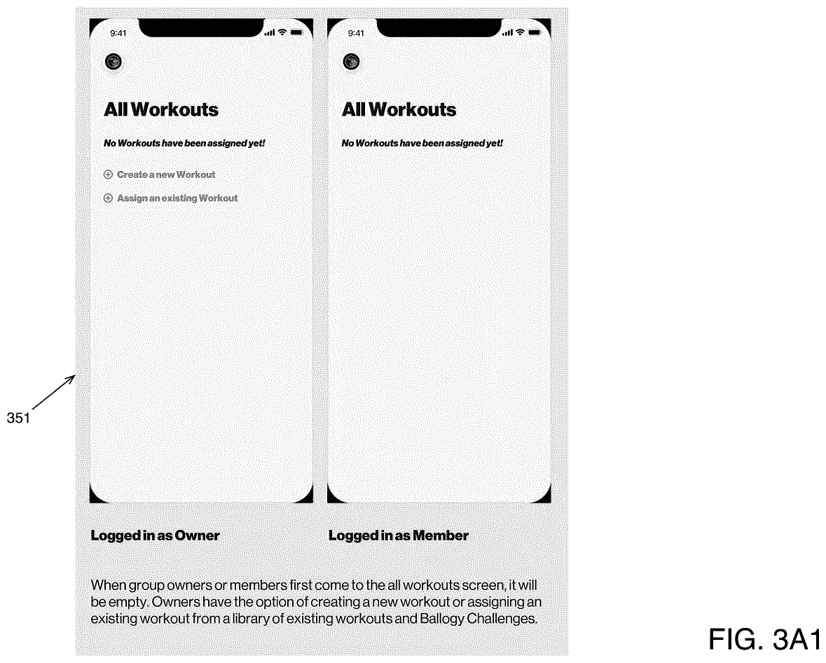

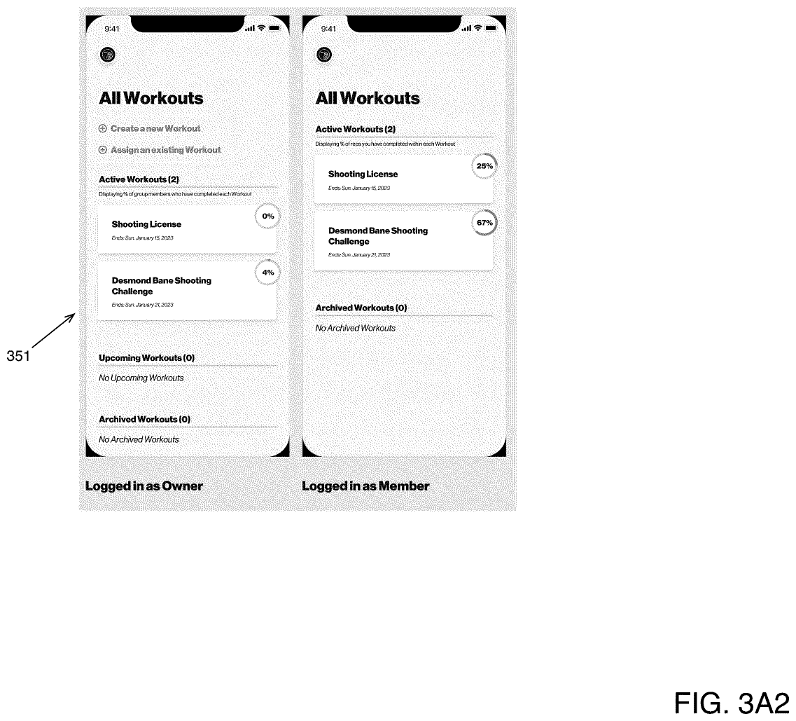

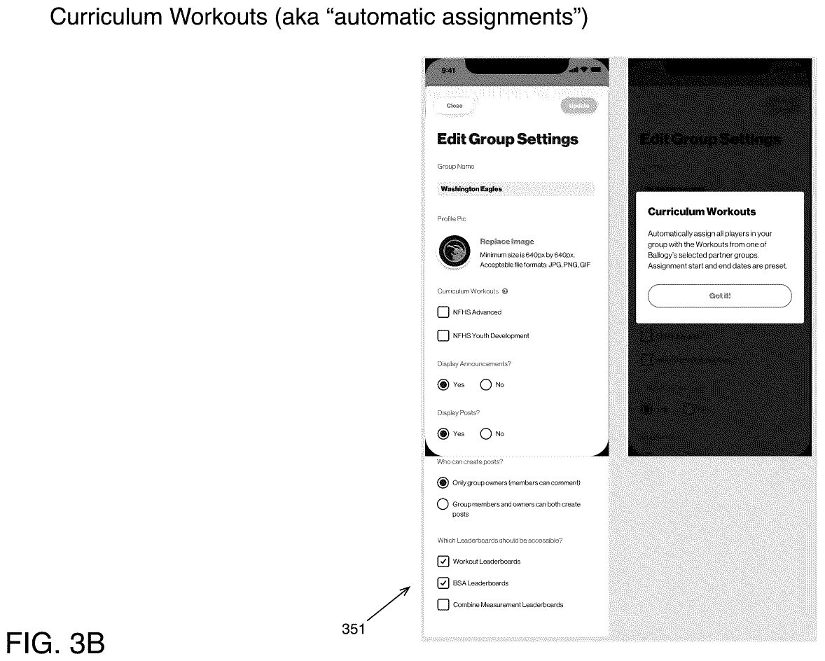

Additional discussion regarding workouts, automatic assignment-type workouts (e.g., curriculum workouts) is provided below. See also the several figures directed to this example subject matter. In the figures, detail is provided with respect to operation of a sports social media application and/or with respect to users' interactions with the app. For example, the discussion that follows may be considered in connection with FIGS. 3-8 and their associated subsections. FIGS. 3-8 depict a numerous functionalities, algorithms, processes, techniques, graphical user interface implementations, etc., that may be self-explanatory. The discussion that follows may include aspects related, at least in part, to aspects depicted in FIGS. 3-8. Of course, subject matter is not limited in scope in these respects.

For example, the discussion that follows may describe example aspects of a “Team Training” and/or a “Create Workout” (or “Curriculum”) functionality of a sports social media application, for example, that may execute, at least in part, on a mobile app, for example. See, for example, FIGS. 3-8.

In implementations, several types of users may be supported. For example, user types may include coaches, players, and/or parents. “Team Training” aspects may be directed more particularly to coaches, for example. Also, for example, “My Workouts” aspects may be directed more particularly to individual users (e.g., athletes). In embodiments, a coach may join a group, for example, and/or may also create Workouts. Individual athletes may also create Workouts, join groups, etc. In embodiments, individual coaches may be provided, via a sports social media application, an access code to enter their group. Players (e.g., individual athletes) may be provided their own access codes to access group materials, again via a sports social media application executing on their respective devices, for example. Groups may also have owners (e.g., coaches, parents) and members (e.g., players, parents), for example.

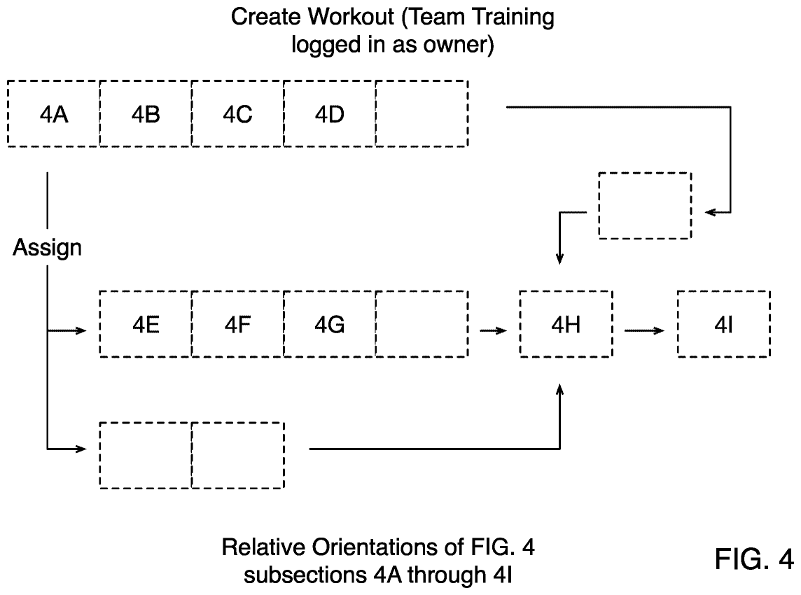

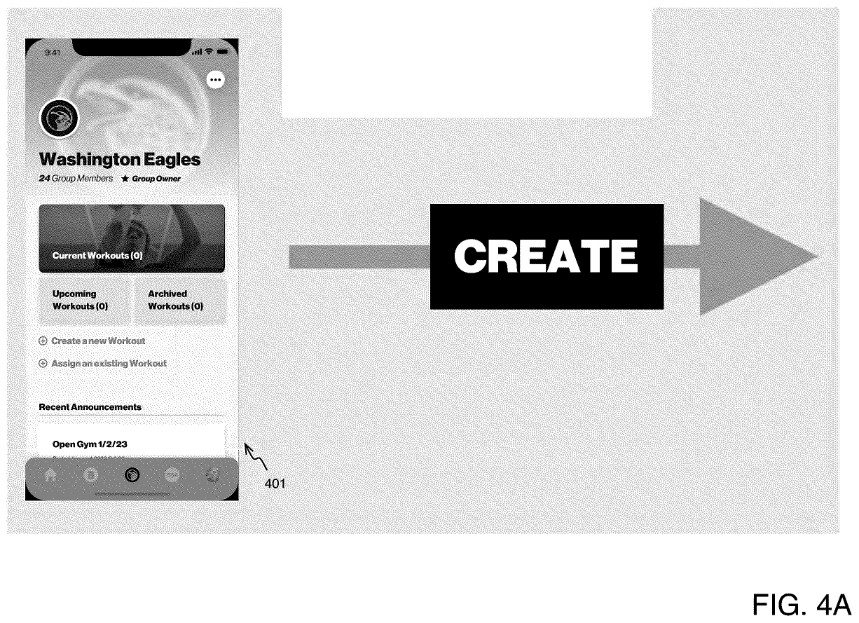

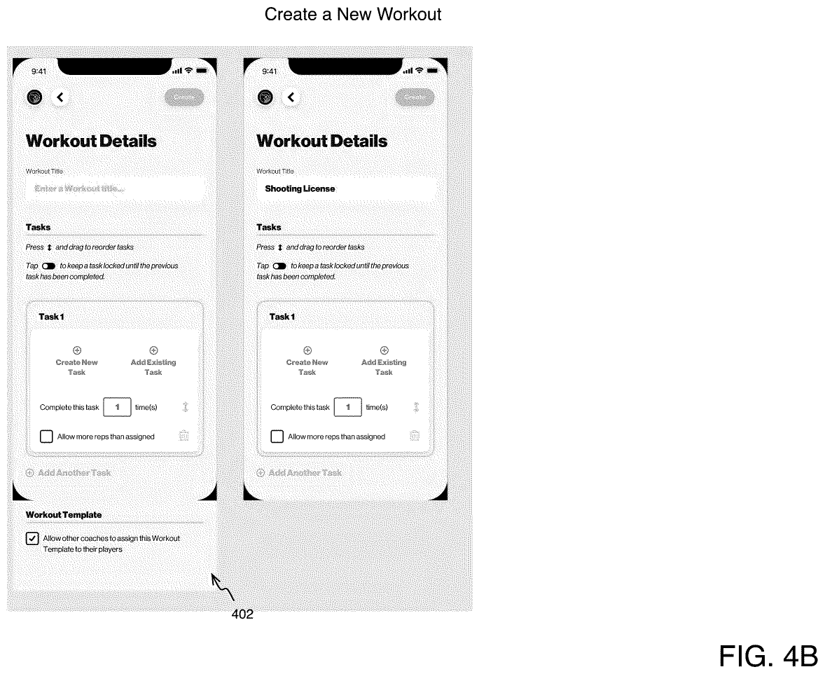

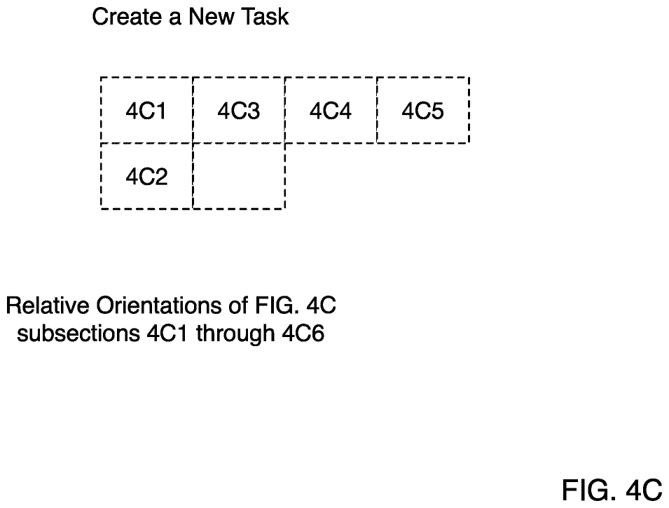

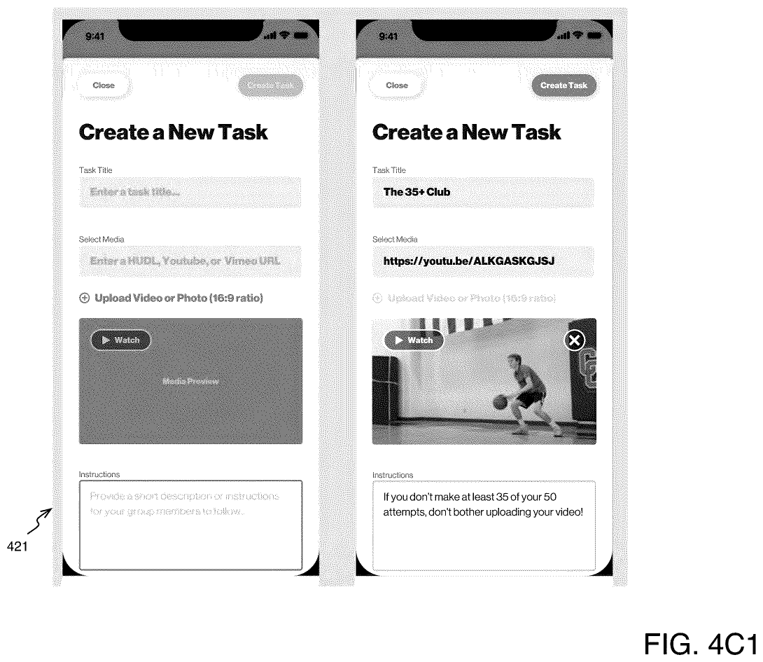

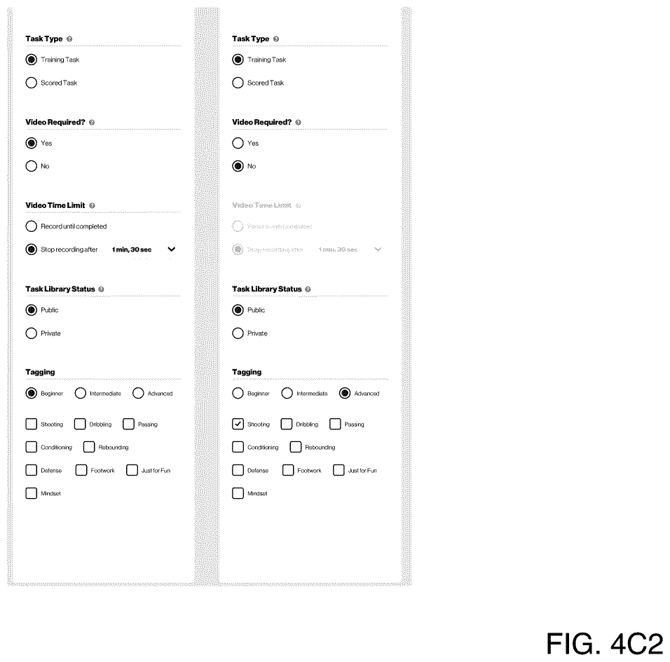

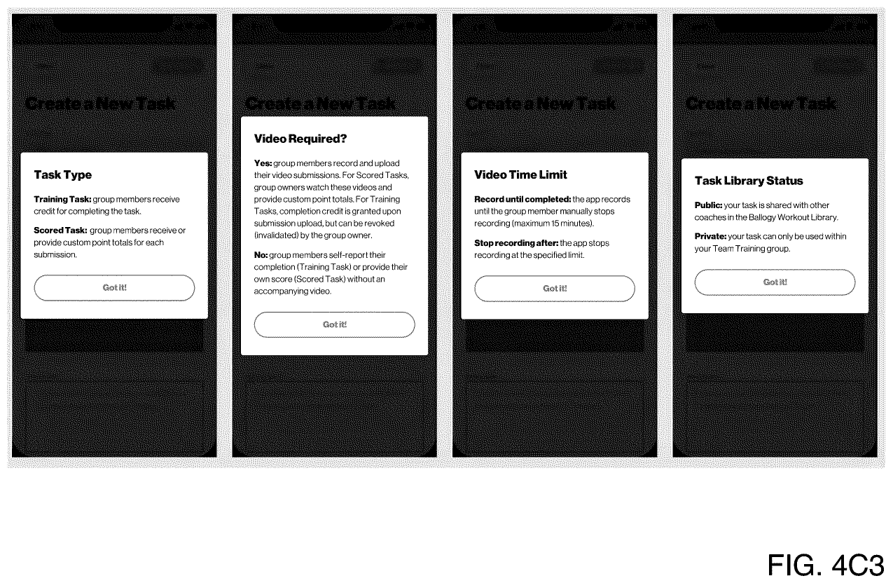

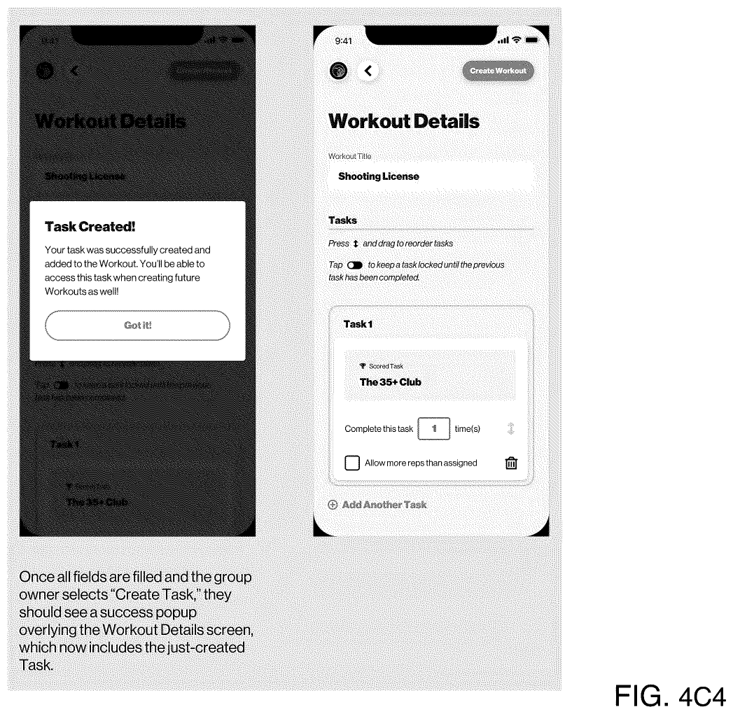

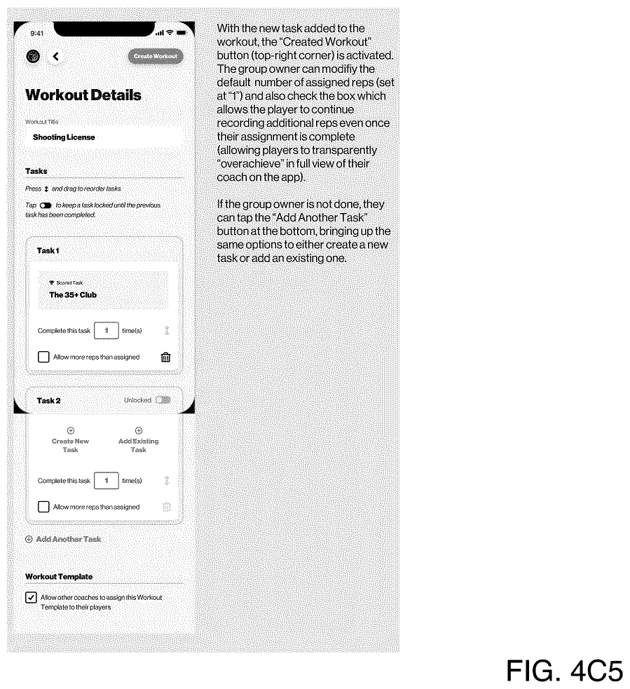

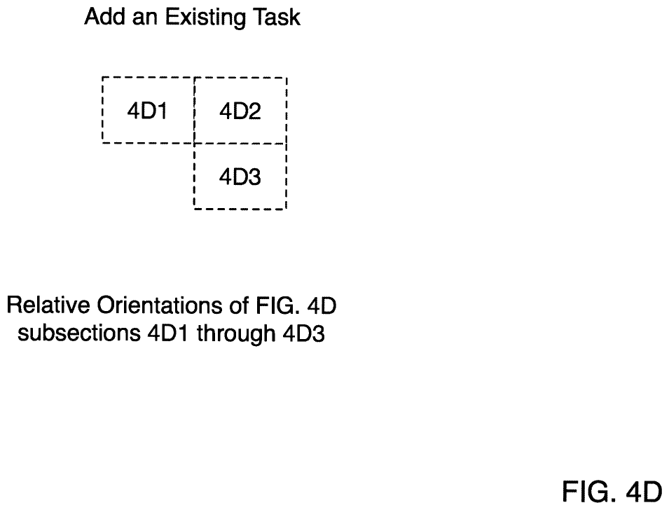

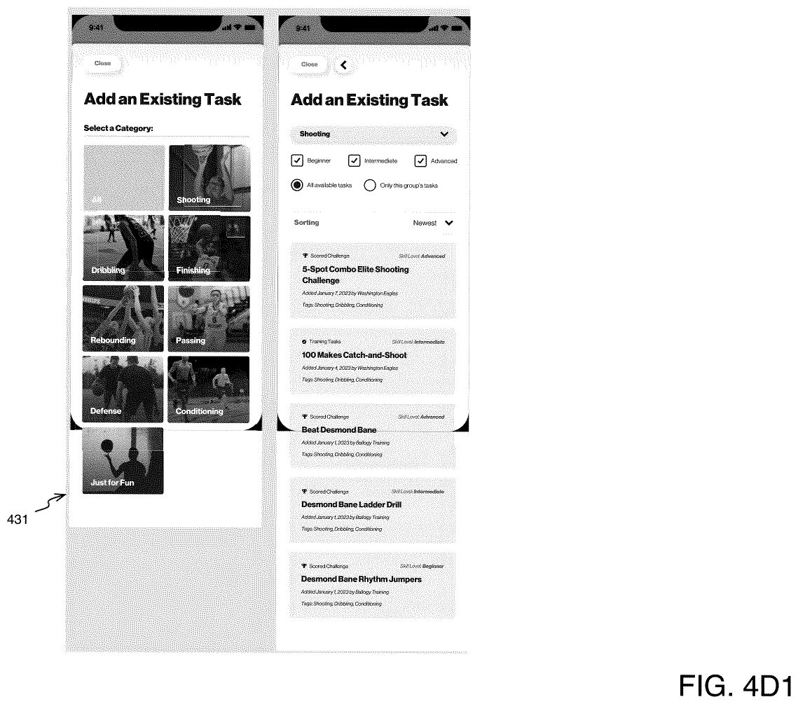

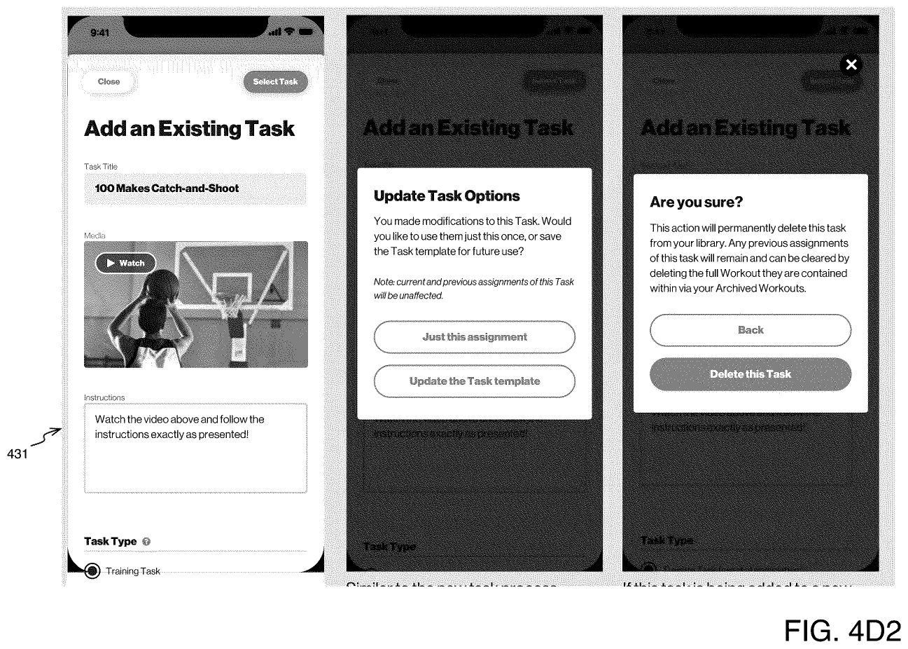

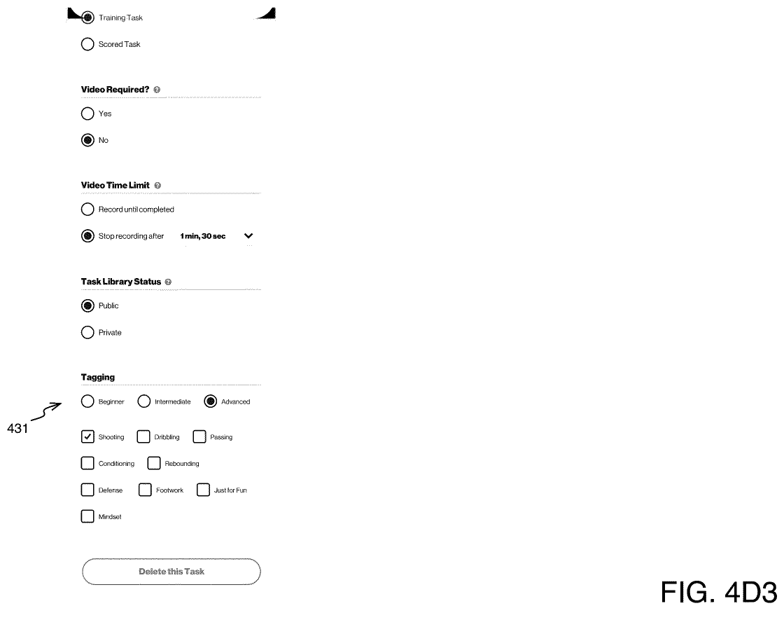

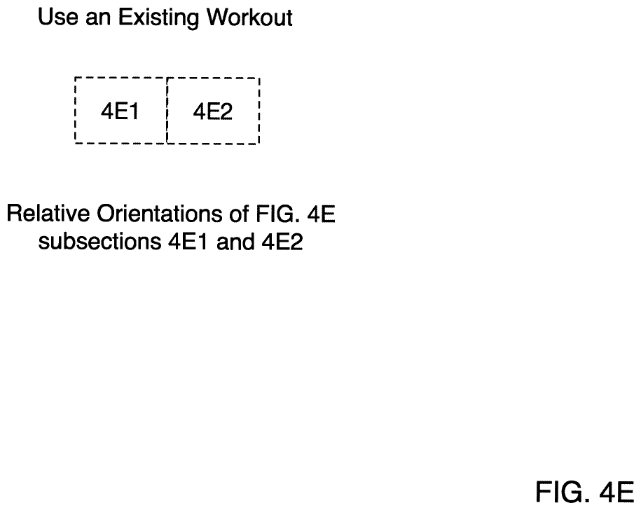

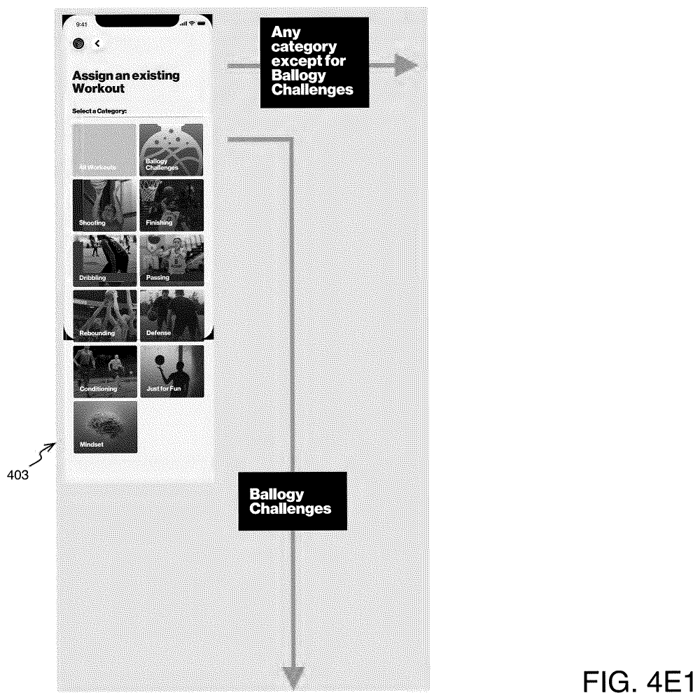

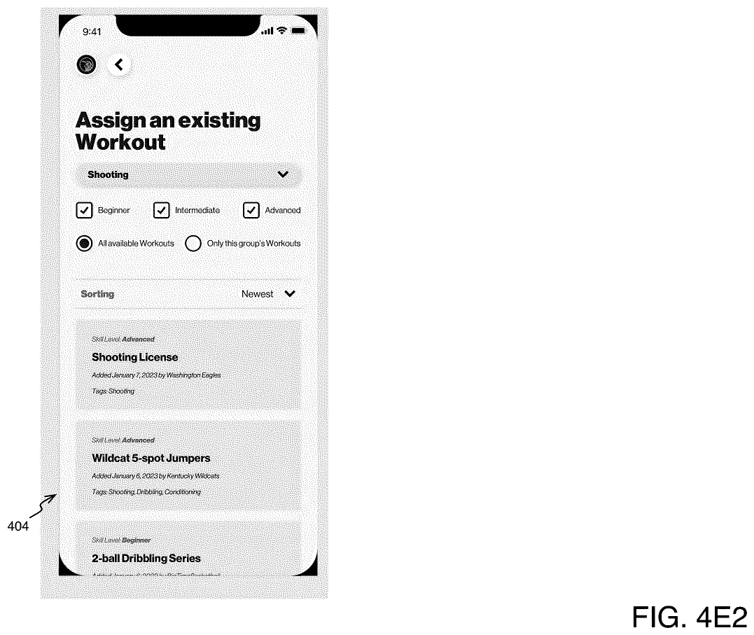

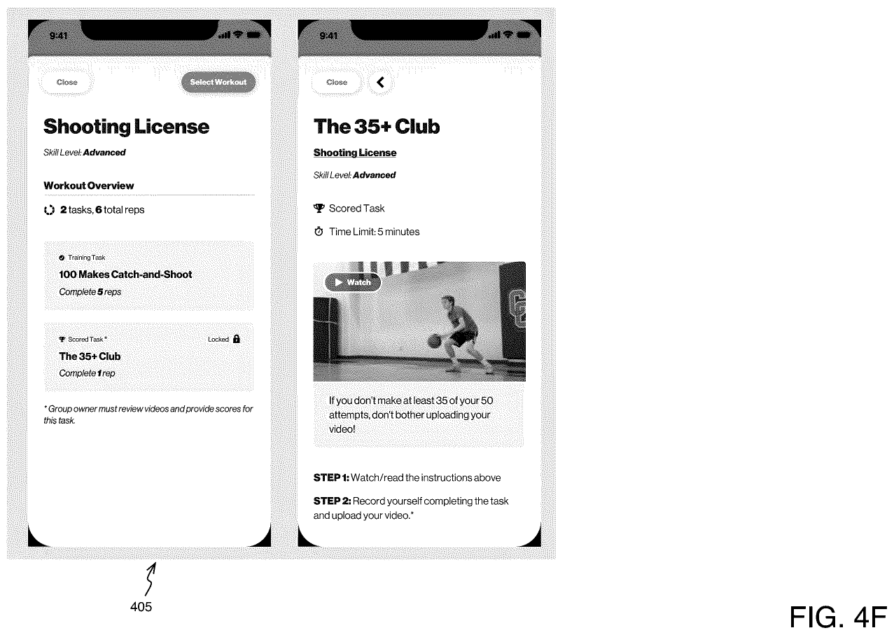

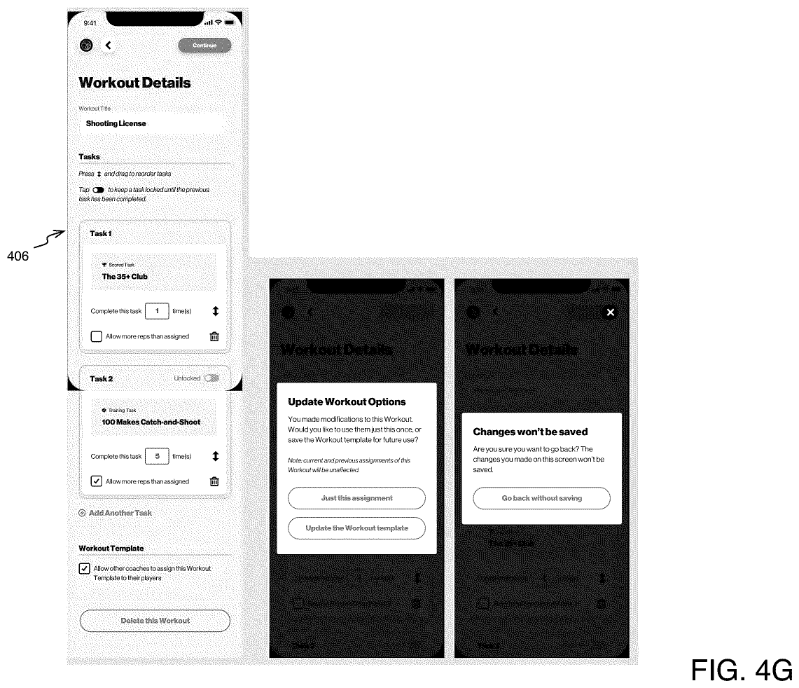

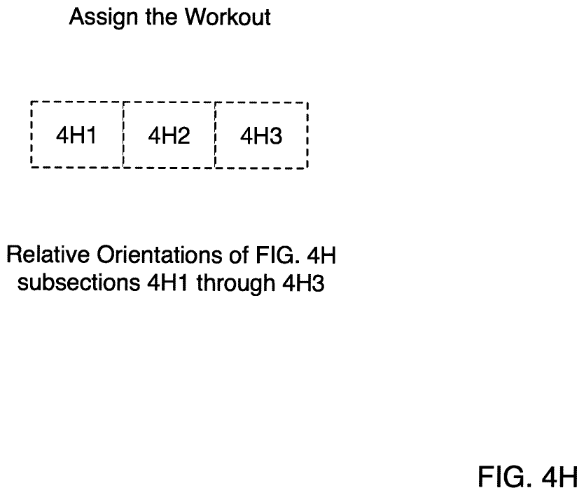

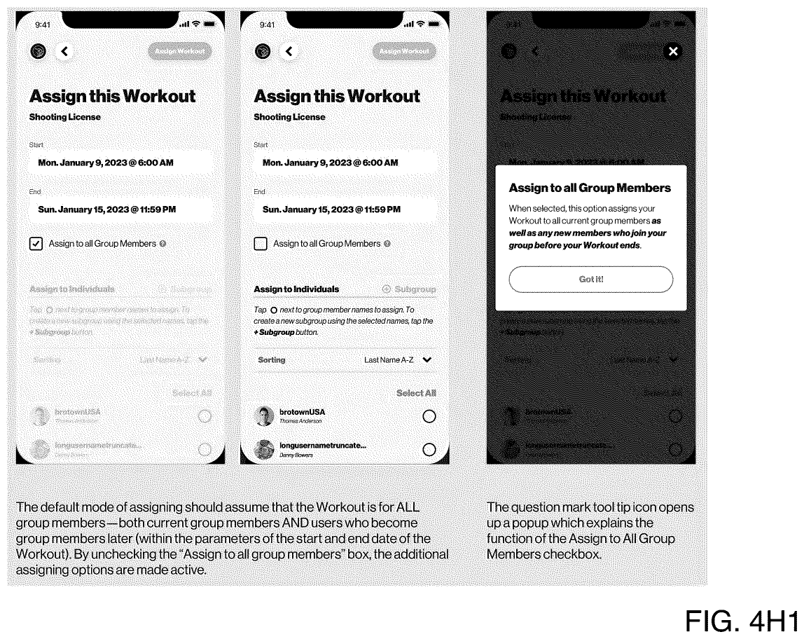

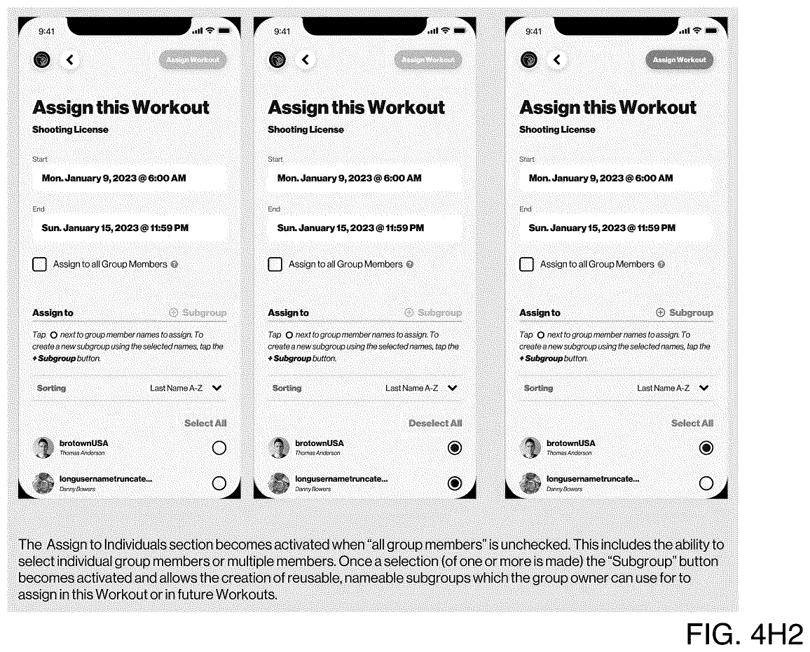

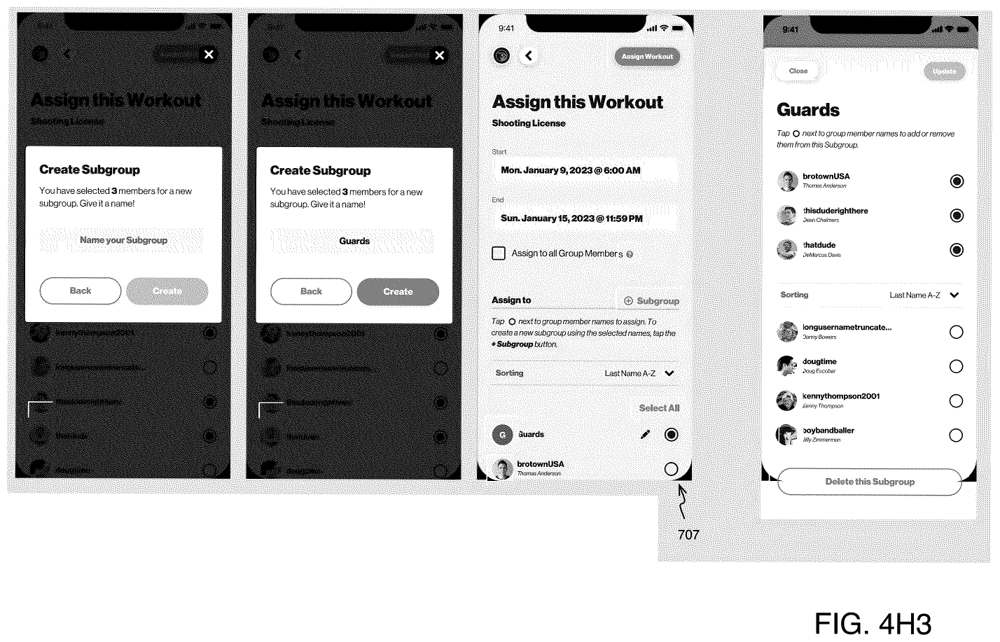

As depicted, for example, in FIG. 4 and its associated subsections, Workouts (e.g., suggested practice routines) may be created and/or assigned from within the app, whereby a coach, for example, may provide instructions or a routine to follow for a team and/or for individual players, for example. Coaches, players, etc. may further upload video content that may or may not be connected with a particular workout. Existing workouts may also be modified, via the app, by users.

As depicted, for example, at example screen 401 of FIG. 4A, group owners (e.g., coaches) may choose to either create a workout or take an existing workout (e.g., created by the coaches and/or by other entities) and assign it to a group in any of several ways, in one or more embodiments. In implementations, a graphical user interface screen, such as screen 402 of FIG. 4B, may allow a coach or other group owner, for example, to create a new workout. In implementations, a shell of a Workout may comprise a title and a series of tasks which may be, for example, placed in a specified order and re-ordered as needed, locked from participation until prior tasks are completed, assigned a specified number of repetitions (“reps”) which may need to be completed before the task as a whole can be considered completed, and/or removed as needed via a trash icon, for example.

In implementations, just as a Workout may be created from scratch or fashioned from previous iterations, individual tasks within such Workouts may also be created from a blank slate or using one that has previously been created (e.g., either by this group owner, other group owners who allow their tasks to be used by others, or by other entities). From example screen 402, a Workout may (e.g., only) be created when the top three fields are completed and at least one task has been added. A “Create” button in the top right corner, for example, of screen 402 may remain inactive until these steps are completed, in one or more embodiments. At the bottom of Workout screen 402 may comprise a “Workout Template” section, for example, which may indicate a public/private status of the workout. In one or more embodiments, if a group owner does not uncheck this box, the Workout will be available for other coaches to use when they select “assign an existing workout” and browse through “all available workouts,” for example.

In implementations, a coach, for example, may select, via the application, any of a range of available pre-defined or existing workouts. For example, workouts related to shooting, finishing, dribbling, passing, rebounding, defense, conditioning, just for fun, mindset, etc. may be available via the app. See, for example, screen 403 of FIG. 4E1. A coach (or other group owner), for example, may select an icon via a graphical user interface of the app to view available workouts for a selected category. In one or more embodiments, Workouts may be categorized as beginner, intermediate, and/or advanced, and a group owner, such as a coach, may select the appropriate level of workout to assign to individual players based, for example, on the individual needs of the player. See, for example, screen 404 of FIG. 4E2.

In one or more embodiments, assigning an existing Workout may start with selecting a category. Workouts may be tagged with multiple categories, so they may be listed in more than one place within the sports social media application. In one or more embodiments, whichever category a group owner selects from the grid of thumbnail images, such as depicted in example screen 403, may become the selected category in the dropdown menu, for example.

In one or more embodiments, a list of Workouts may feature all of the Workouts previously created by a particular group and/or all Workouts created by other groups if such groups indicate an option to make their Workout public. In one or more embodiments, by default, a group owner may see “All available workouts” in this list, with the option of reducing this down to only the Workouts they created themselves by changing the radio button selection, as depicted at example screen 704.

In implementations, workouts may be created and/or modified by coaches, for example. The application may provide an interface whereby the workout creator/modifier may elect to keep the workout private (e.g., available only specified individuals) or may elect to make the workout available to others by selecting a “public” option, for example. Further, for example, coaches (or other group owners, for example) may preview workouts prior to assigning them. For example, a coach may view a portion of a workout (workouts may comprise video content) to assess its applicability and/or appropriateness before assigning the workout to a player. See, for example, screen 405 of FIG. 4F and screen 406 of FIG. 4G.

As further indicated at screen 405, tapping on any of the existing Workouts results in a popup which slides up from the bottom of the screen, in one or more embodiments. This screen may allow a group owner to preview a Workout and any or all tasks within it, for example. In one or more embodiments, tapping a “Select Workout” button may advance a user to make final adjustments to a Workout before assigning it to the group. If the original Workout was created with a task using the manual Scored Task option (in which the group owner watches videos and provides a score), this designation may be noted on a main Workout screen, for example.

In one or more embodiments, after selecting a specific workout, example screen 706 may become a starting point for creating a Workout quickly. In one or more embodiments, a group owner may be allowed to make edits to all fields, including removing, re-ordering tasks, and adding new tasks, for example. Alternatively, they can simply tap “continue” if no changes are needed, in one or more embodiments. In one or more embodiments, if any changes have been made to this screen (e.g., adjustments to the tasks or changes to the title), a popup message may allow a group owner to choose between using these changes for this one workout assignment or saving it for all future uses, for example. In one or more embodiments, if changes have been made and the owner chooses to go back to the previous screen or the group home screen, a popup warns that the changes will not be saved, in one or more embodiments.

In one or more embodiments, Workouts may include tasks for a player to perform, and a coach may specify a number of repetitions for the player to perform, for example. A coach may also modify a sequence of activities for a player to perform, for example. As shown in the various example screens of the various figures, a number of customization options may be provided via the app. In implementations, a coach or other group owner may specify particular aspects of a workout, but may be prevented from altering the contents of a workout. For example, if a workout includes a shooting drill, a coach may specify a particular number of repetitions for a player to perform, and/or may specify a conditioning drill to be performed prior to the shooting drill, for example, but may be prevented from altering the contents of the shooting drill itself.

Once created, a workout may be assigned, again via the app, to a single player, to a group, or to a subset of a group, for example. Start and end time and dates may be specified, for example. See, for example, screens 407 of FIG. 4G3.

Once assigned, a workout may be viewed by a coach, for example, via an “All Workouts” screen of the app. See, for example, screen 601 of FIG. 3A and/or screen 408 of FIG. 41. This screen may show which workouts are currently active and when they expire. Upcoming and/or archived workouts may also be shown, and options for creating a new workout or assigning additional existing workouts may also be provided.

Additionally, functionality may be provided by the app to automatically generate “Curriculum Workouts” (may be referred to as “automatic assignments”) as shown at screen 351 of FIG. 3B, for example. In embodiments, coaches may assign existing curriculum Workouts and/or may assign existing Workouts. These automatic assignment-type Workouts, selectable via a sports social media application, may provide a systematic and/or customized approach to training, for example. In embodiments, coaches may be assured that players following such curriculum Workouts will improve during the offseason, preseason, or in-season. That is, for example, curriculum Workouts may be automatically generated based on offseason needs for a player or group, for pre-season needs, and/or for in-season needs. In embodiments, start and/or end dates may be specified, such that a coach will not need to set new tasks, or create additional Workouts, or the like while the curriculum Workouts are active. Generally, players may be trained during these different phases of the year or season via their interaction with a sports social media application. That is, for example, a sports social media application may be thought of as training the players on behalf of a coach. Of course, a coach may design the type of program they might like their players to follow, but the details of the Workouts and assignment, as well as tracking, teaching, and reporting, for example, may be handled via a player's interaction with a sports social media application, in embodiments.

As an example, referring again to FIG. 3B, “NFHS Advanced” and/or “NFHS Youth Development” curriculum Workouts may be selected. Different curriculum Workouts may have particular shooting, finishing, dribbling, passing, rebounding, defense, conditioning, just for fun, and/or mindset, etc. Workouts as part of their particular plans for various phases of the year (e.g., preseason, offseason, in-season, etc.). Such automatic assignment-type Workouts may be designed by coaches or other experts to guide players through offseason development, preseason development, and/or in-season development, in embodiments. In embodiments, start and/or end dates may be pre-defined, so the coach wouldn't need to be concerned with that aspect. As depicted in FIG. 3B, curriculum Workouts may be assigned on a group basis, in some implementations. In embodiments, players in a group may be assigned these Workouts on behalf of a coach, for example, and/or coaches may select appropriate curriculum Workout for their particular groups.

In implementations, a sports social media application may designate a particular routine, such as a shooting routine, passing routine, dribbling routine, etc., or a combination thereof, as part of an automatic assignment-type Workout. In embodiments, a group that is assigned to a particular automatic assignment-type Workout may be assigned automatically assigned a particular routine. For example, a sports social media application, perhaps being executed at a server computing device, may push an indication of this new assignment to members of groups that have been assigned (e.g., by their coaches or other owners) a particular assignment-type Workout. In other words, rather than a coach going through the process of selecting and/or creating Workouts described previously, such assignments may be made automatically in accordance with a selected curriculum Workout managed automatically via a sports social media application, in embodiments.

Once the new routine/workout has been assigned as part of a curriculum/automatic assignment-type workout, individual players belonging to groups subscribing to the particular curriculum/automatic assignment-type workout may receive a message, such as via a pop-op alert on their individual electronic devices, and the individual players may then access the new routine/workout via the “All Workouts” screen of the app (e.g., screen 351 of FIG. 3A and/or screen 408 of FIG. 41), for example.

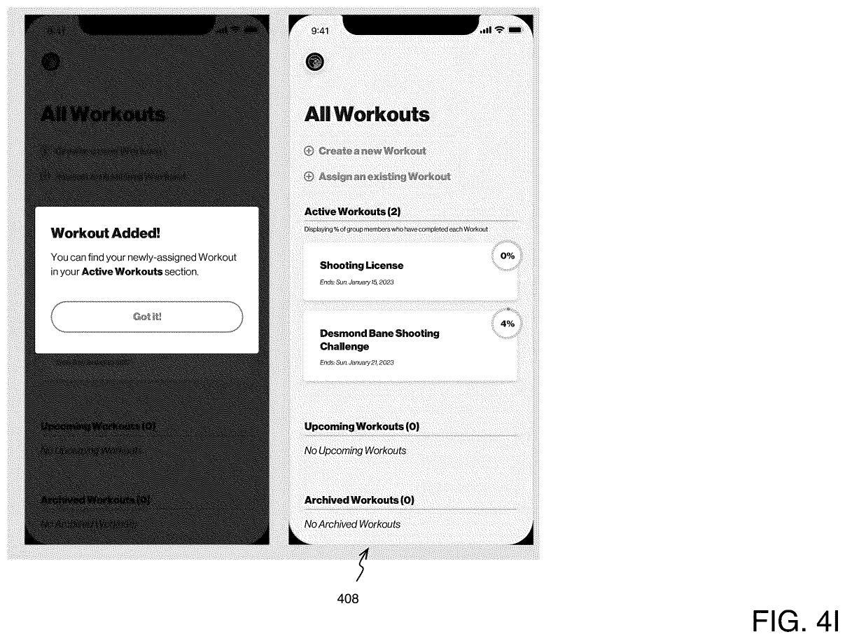

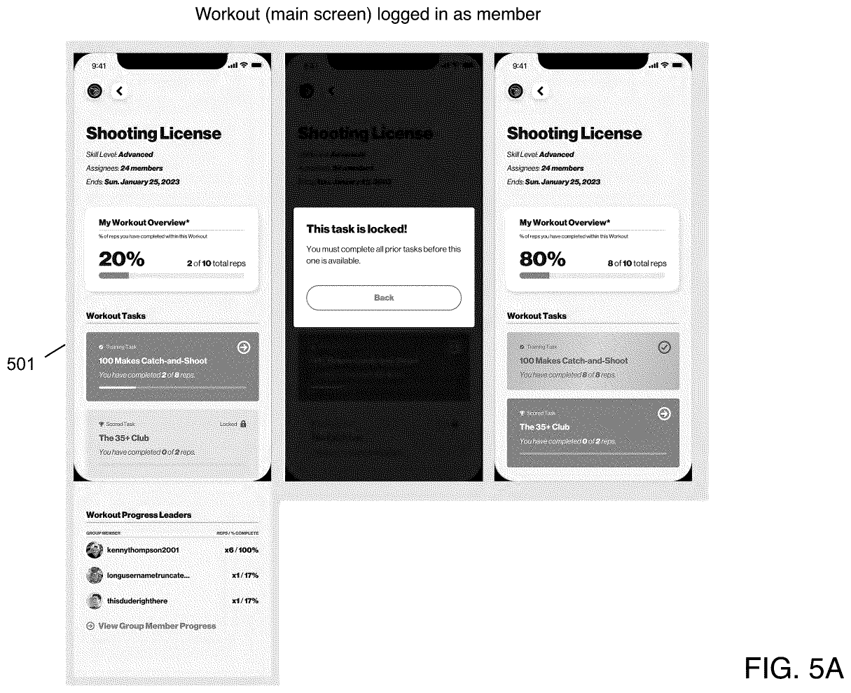

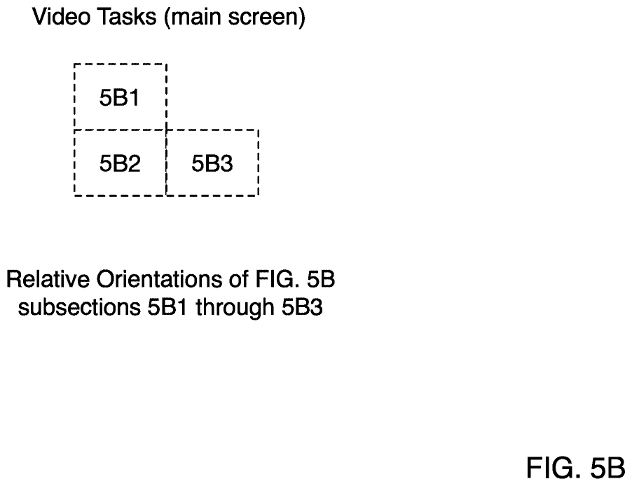

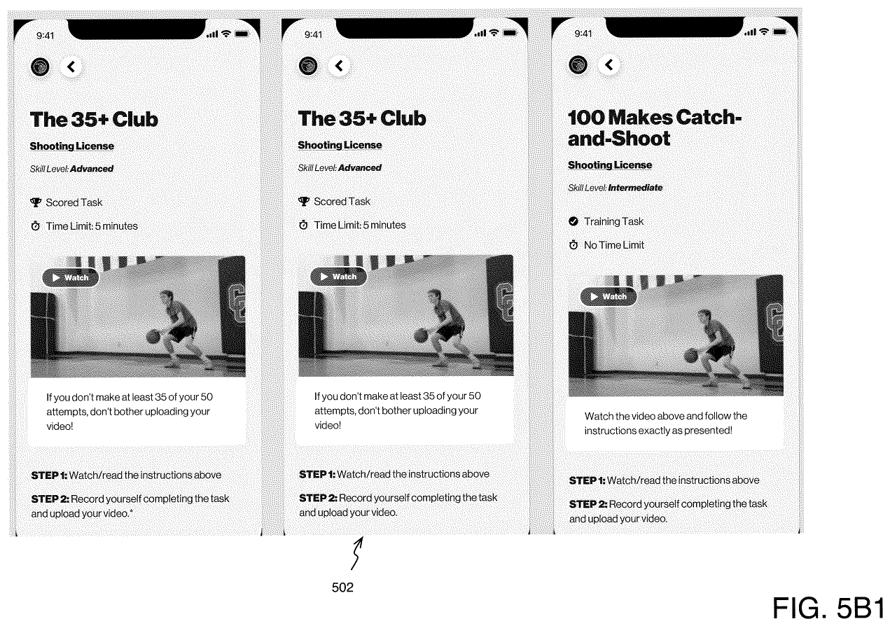

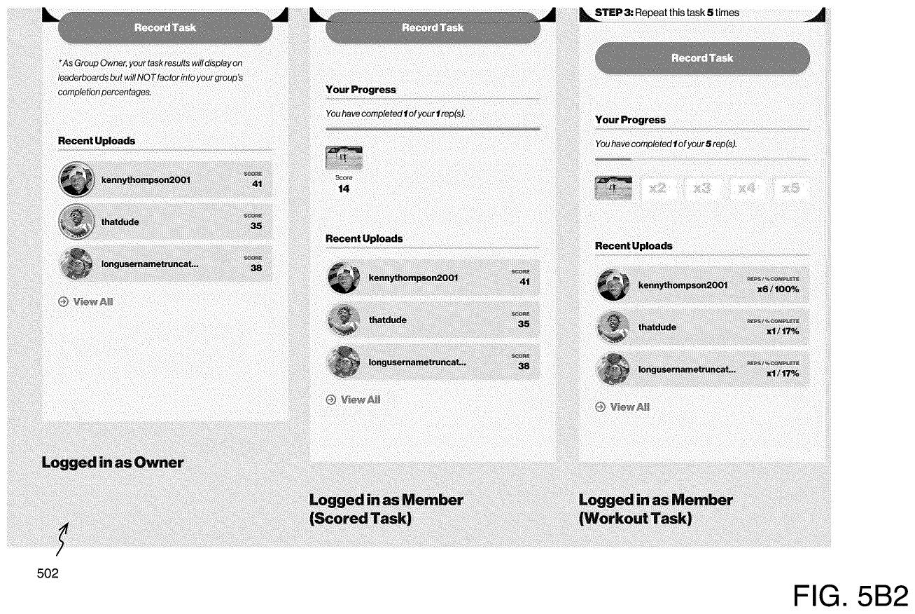

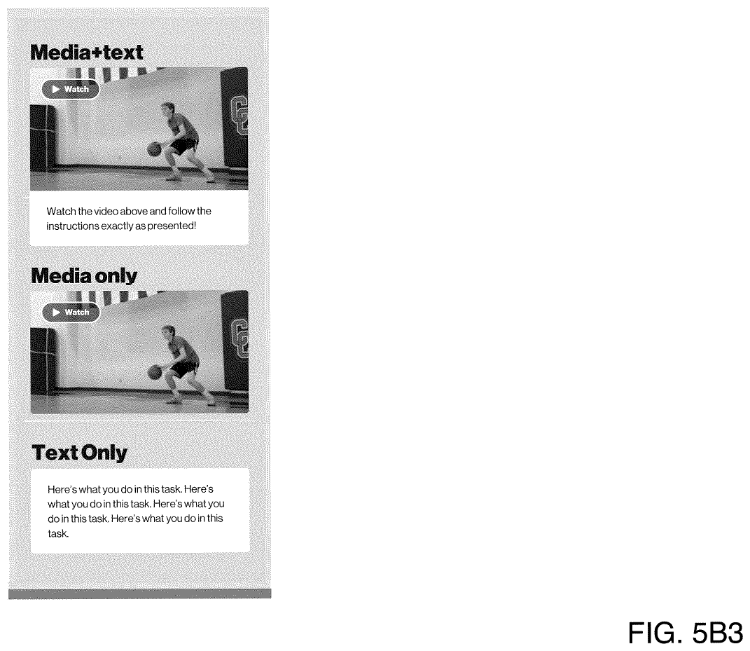

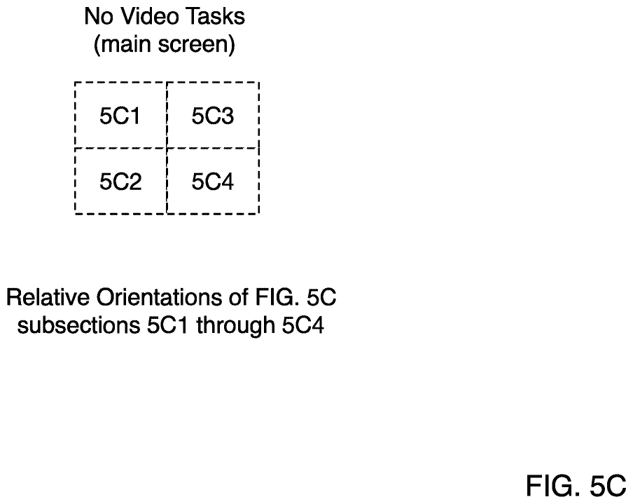

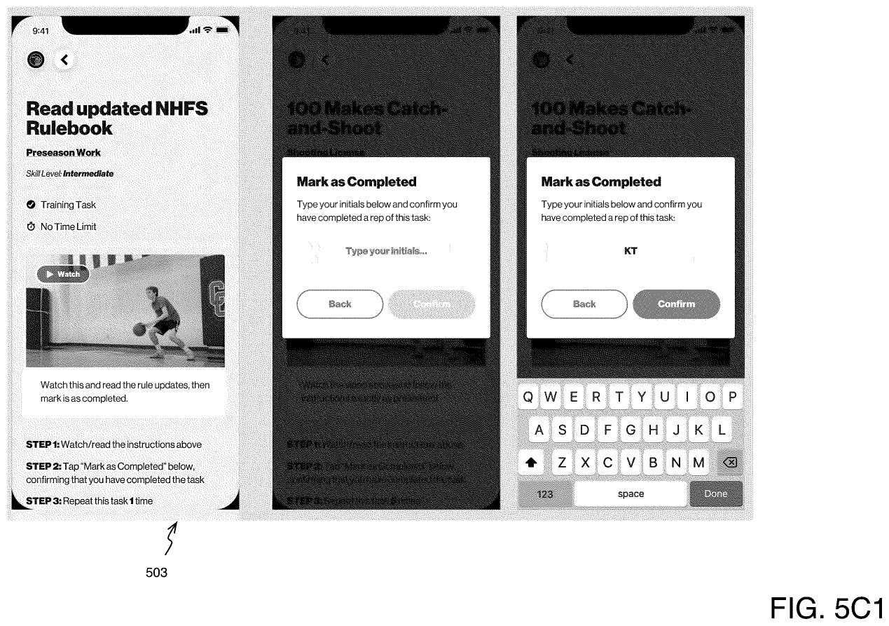

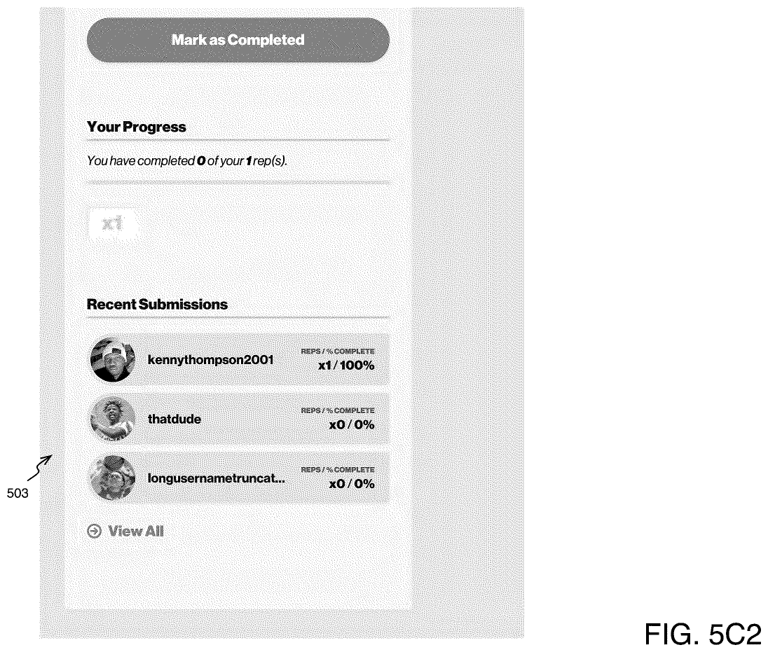

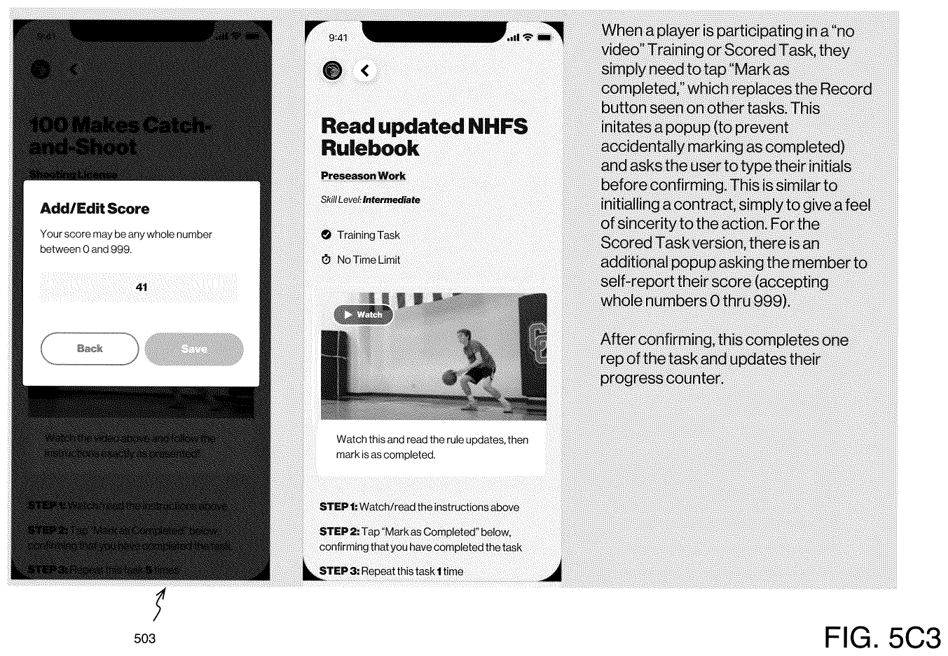

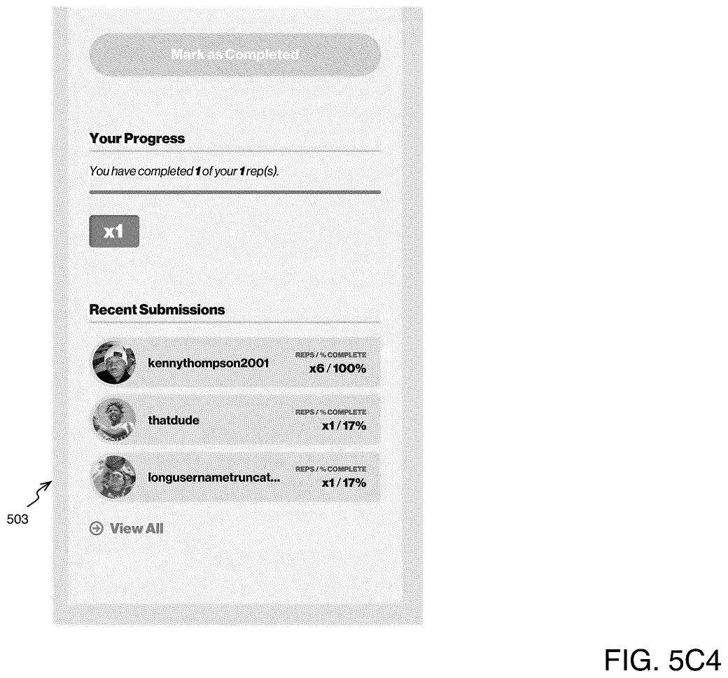

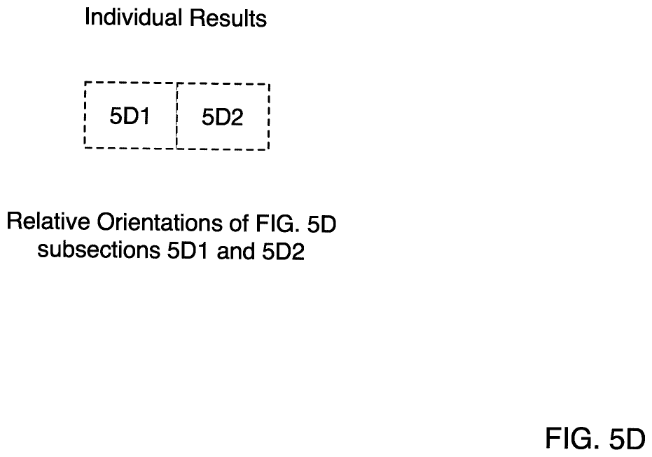

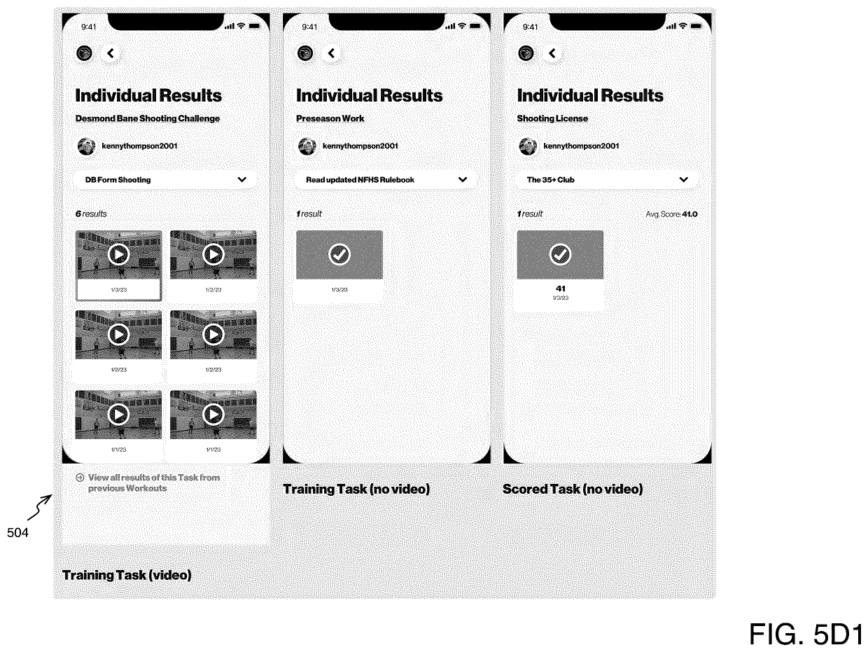

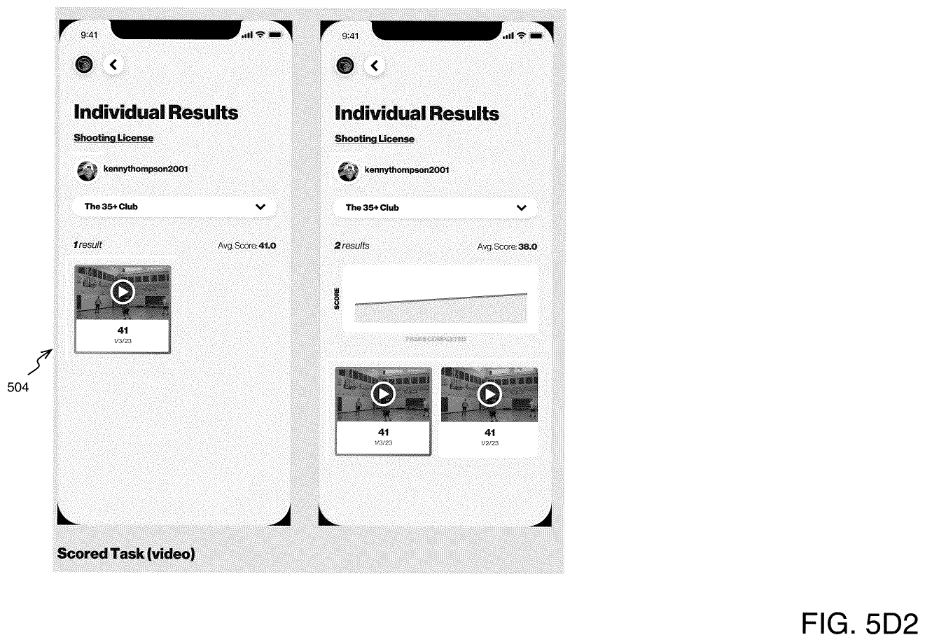

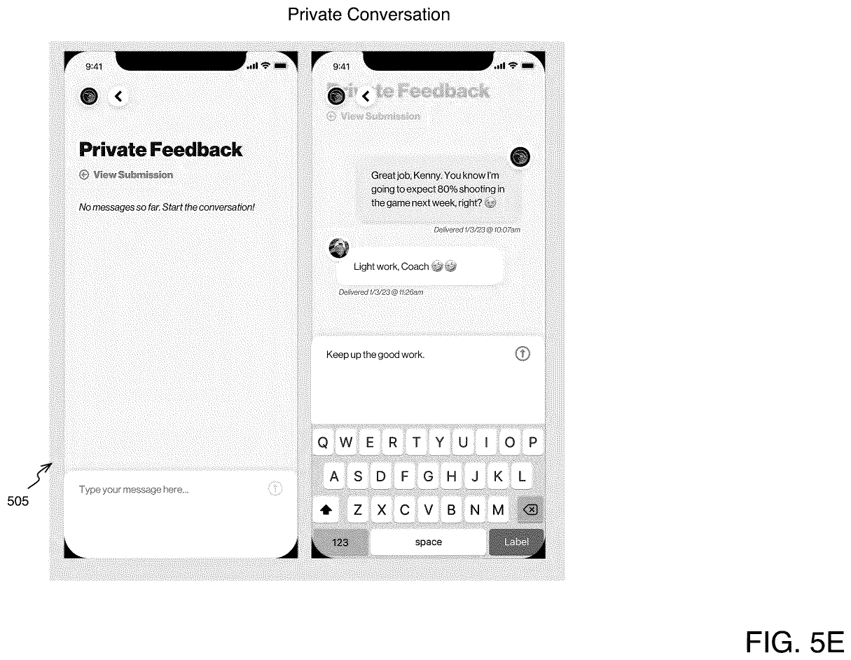

In embodiments, responsive to a user, such as a player, for example, selecting a particular Workout via a sports social media application, the app may display to the user a page that may communicate various aspects of the assigned Workout. For example, the specific drills to be completed, the number of reps to be completed, etc. may be displayed. Further, a sports social media application may track, such as via video input as the user performs the assigned tasks, the user's progress in completing aspects of the assigned Workout. See, for example, screen(s) 502 of FIG. 5B.

In embodiments, a sports social media application may direct a user to a screen showing information regarding tasks to be completed. For example, a sports social media application may display video content to the player (or other type of user) via the user's electronic device (e.g., tablet, cell phone, etc.) to demonstrate to the player how to execute the task. For example, responsive at least in part to a user selecting a particular element of a particular screen in a sports social media application, the app being executed on the player's electronic device may communicate with a server computing device, for example, and video content may be communicated to the user's electronic device and displayed to the user. Various communication, server, and/or network technologies are described herein.

Additionally, in embodiments, a sports social media application being executed on the player's electronic device may provide a user interface element whereby the player may initiate a video recording. See, for example, screen(s) 502 of FIG. 5B. In embodiments, a video recording may be made via a sports social media application to record the player executing the assigned task. In embodiments, recorded video content may be analyzed in real-time or near real-time via AI and/or other machine-learning techniques, for example, and/or may be provided to remote computing devices to enable analysis by other devices or individuals (e.g., coaches). Also, in embodiments, a coach may designate a particular Workout as “non-video” or the like to indicate that the player is not asked to provide a video recording. See, for example, FIG. 5C. In implementations, a sports social media application may grey out the recording feature of the user interface in such circumstances. Alternatively, for a no-video task, a sports social media application may display to a player a means for the player to manually enter the player's progress (e.g., “completed” and/or the like). If it's a scored task, the player may add or edit their score via the user interface of a sports social media application, for example. See screen(s) 503 of FIG. 5C1, for example.

In implementations, once a player, for example, uploads a video, the video may become available via the app on a player's “individual results” screen. See, for example, screen(s) 504 of FIG. 5D. Also, for example, an individual results screen may associate a score, such as may be generated by the app using AI/machine-learning analysis (e.g., including communication between a user's electronic device and a server computing platform) and/or by a coach's analysis, with an uploaded video. A coach, for example, may analyze a player's uploaded video and may determine a score to assign to the uploaded video. That is, a coach may analyze a player's performance of the assigned workout using the player's uploaded video. As mentioned, such analysis may be performed by AI/machine-learning analysis in some implementations.

Also, in embodiments, a sports social media application may provide instructions to a coach on how to score particular Workouts. In this manner, scoring from one coach to another may be standardized to a large extent. In embodiments, some Workouts may not involve scored Workouts, but instead may be designated as non-scored. In this circumstance, if it is a video-recorded Workout, a coach may simply indicate that a particular Workout has been completed, as evidence by the player's uploaded video, for example. As mentioned, some Workouts may ask the player to self-report. In the case of a coach-scored Workout, the player may receive a notification, via a sports social media application, that a coach has scored the Workout, for example.

In implementations, feedback may be provided to the player for completed workouts. See, for example, screen(s) 505 of FIG. 5E. The app may provide a chat feature whereby a player and coach may communicate with each other.

As mentioned, for some workouts a player may be asked to provide a score for a completed task. As shown in FIG. 5C1, for example, the app may display to the player guidelines for scoring an assigned workout. For a training task, for example, a player may tap a “mark as completed” button on a graphical user interface (screen) and/or may be asked to enter the player's initials via the graphical user interface, for example. For scored workouts, the player may self-assess in accordance with provided guidelines or instructions provided by the app and the player may enter a score. As also mentioned, workout analysis and/or scored task analysis and/or scoring may be accomplished via AI/machine-learning implementations, which may include communication between a server computing device and a user's device, for example.

As mentioned, coaches or other users may assign Workouts to players or groups of players, for example. In embodiments, a coach, may for example, design their own training program for one or more players by selecting pre-existing Workouts to assign to a group or individual. As also mentioned, curriculum Workouts (automatically-assigned Workouts) may be selected by a coach to assign to groups or individuals. With curriculum-type Workouts, a coach may design their own based on existing Workout routines in the various available categories (shooting, passing, defense, conditioning, etc.) and may manually manage the assigned Workouts throughout the course of a cycle (e.g., off-season, preseason, in-season). A coach may also assign app-generated and/or managed curriculum to individuals or groups, for example. In this case, a sports social media application may automatically assign particular Workouts in the various available categories as appropriate given the cyclical nature of the basketball sporting calendar, for example (of course, subject matter discussed herein may be expanded to include other sports or skilled activities). For example, a sports social media application may assign particular Workouts in the offseason, may alter assigned Workouts as the offseason progresses, may again alter assigned Workouts in the preseason phase, and/or may further adjust assigned Workouts during the in-season phase. In implementations, a coach need not manually manage these aspects for curriculum-type Workouts.

Also, for automated curriculum-type Workouts (e.g., offseason, preseason, in-season cycle), assigned Workouts may be adjusted based on individual player's progress and/or development as determined either by a coach and/or as determined via AI/machine-learning analysis, in embodiments. In embodiments, both a coach and a player may monitor the player's progress, whether that progress be evaluated by a human coach or whether evaluated via AI/machine learning.

As an example, basketball tends to be cyclical and generally fixed in terms of off-season, preseason, and/or in-season. A sports social media application may be implemented to understand that from late August/early September to the end of October may comprise a preseason and that October going into November is generally when the basketball season starts for every single high school amateur team across the country in the United States. The offseason may be considered to start in April and may continue until the start of the preseason, for example. A sports social media application may be programmed with particular start and/or end dates for the various phases of a sporting calendar. Also, a sports social media application may also track start and end dates for the various cyclical stages of a calendar for a given sport for any number of different schools, leagues, organizations, etc.

In embodiments, a sports social media application may automatically assign Workouts, tasks, etc. based on this cycle or coaches may assign Workouts as they see fit. For example, a sports social media application may generate a particular Workout curriculum for the preseason phase. A coach may select particular Workouts from that curriculum to assign to players, or a coach may assign an automated curriculum (automatically assigned Workouts) which may be managed by a sports social media application, wherein a sports social media application automatically assigns particular Workouts, tasks, etc., depending on whether it's the offseason, preseason, in-season, for example. See FIG. 4 and its associated subsections for more self-explanatory details regarding app functionality. Workout plans may also automatically be altered during a particular phase of the calendar year, in some implementations. In embodiments, different Workouts may be automatically assigned on particular days depending on a team's specific schedule, for example, or a coach may assign particular tasks, Workouts, etc. depending on a coach's team's schedule. A coach may also opt-in to individual Workouts and/or a Workout plan as they deem it advantageous for individual players and/or groups (e.g., teams), for example.

In implementations, a Workout plan, such as an automated curriculum plan and/or a coach-generated plan, may include start and/or end dates for when particular Workouts “go live.” A sports social media application may monitor time and date and may automatically assign a particular Workout to appropriate player(s) when a specified time and/or date is reached. For example, a sports social media application or a coach may designate a particular Workout to be assigned to a group at a particular time and/or date. When that time and/or date arrives, a sports social media application automatically assigns the Workout and the Workout may be visible to the player(s) via a sports social media application (e.g., a sports social media application or portion of a sports social media application being executed on their personal electronic devices, such as a cell phone, tablet, etc.). The player may be notified via a pop-up display on the player's electronic device, for example.

Restating some of the above, the app may automatically generate a workout plan (“curriculum” plan) that may include a number of workouts from various categories, such as those depicted in screen(s) 431 of FIG. 4D1, for example. In embodiments, automatically generated plans may change during the course of a sporting cycle, such as basketball's general offseason, preseason, in-season cycle, for example. Various Workouts included in a curriculum plan may be automatically assigned to players at appropriate times and/or dates. Also, for example, coaches may select a subset of curriculum plan Workouts to assign to individuals or groups, or may add Workouts to a curriculum plan. Coaches may also design their own curriculum plans, for example. One advantageous aspect of sports social media application is that many of the arduous tasks involved in coaching may be transferred from a coach to the app, and may help more efficiently and/or effectively develop players and/or teams. AI/machine-learning analysis may add to these advantages by helping coaches evaluate players, Workouts, tasks, drills, etc. and/or by providing timely feedback to players as they work to perfect their techniques and skills. A sports social media application may also help organize a coach's efforts to personally guide players and teams in their development, for example.

As mentioned, in some implementations, an AI/machine-learning analysis engine may analyze a player's uploaded video content, for example, to score a task or Workout and/or to analyze a player's technique, movement, skill completion, etc. In implementations, a sports social media application may also include a recommendation engine that may, based at least in part on AI/machine-learning analysis, recommend to a coach, player, parent, etc., particular Workouts to be assigned to the player to develop the player's performance. Such recommendations may be customized for individual players based. Also, a recommendation engine may rely not on AI/machine-learning analysis of video content, for example, but may instead, or in addition, base the recommendations on a player's results for a particular assessment, Workout, task, etc.

For example, a Ballogy Skills Assessment (BSA) may measure a player's ability to make a specified number of basketball shots from specified areas of the basketball court in a specified order in a specified period of time, as explained previously. A recommendation engine may obtain results of such an assessment, with or without accompanying video, and may recommend particular Workouts for the player based on the assessment results. For example, if a BSA shows a particular player missing a relatively larger number of shots in a particular region of the basketball floor (e.g., 3-pt line, right left wing), a sports social media application may recommend Workouts designed to improve a player's skill in making shots from that region of the floor. As mentioned, recommendations may also be based, at least in part, on AI/machine-learning analysis of a player's techniques, skills, movement, etc., as evidenced by recorded video content, for example.

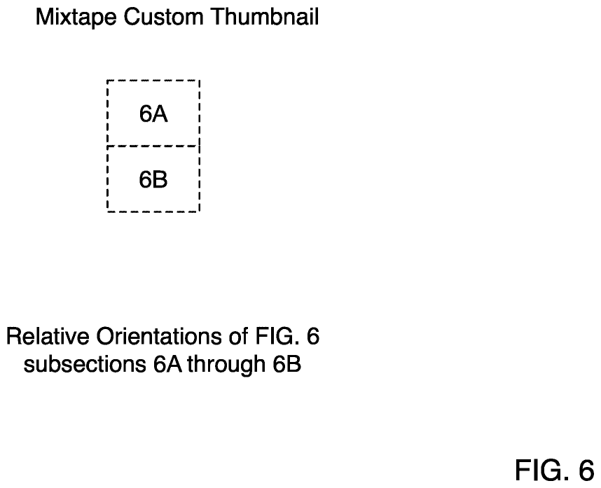

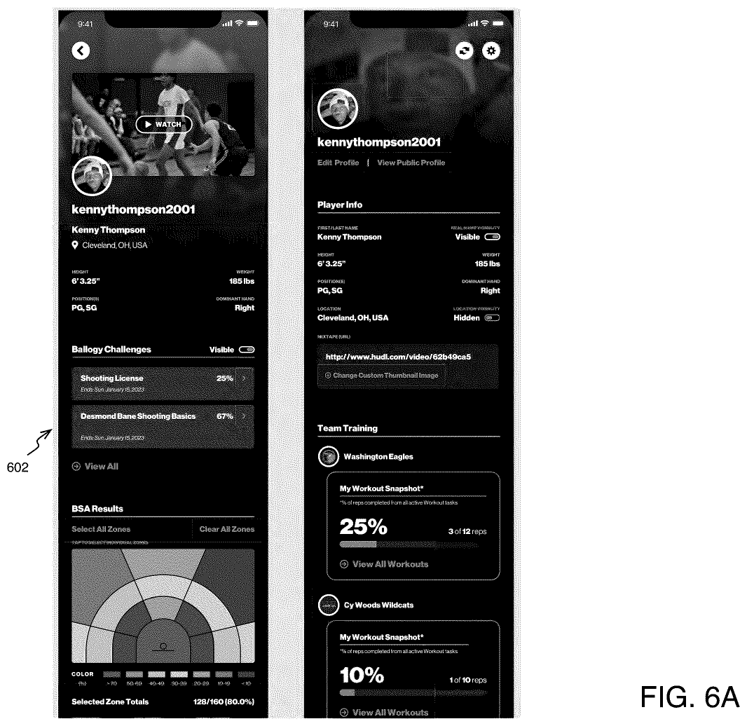

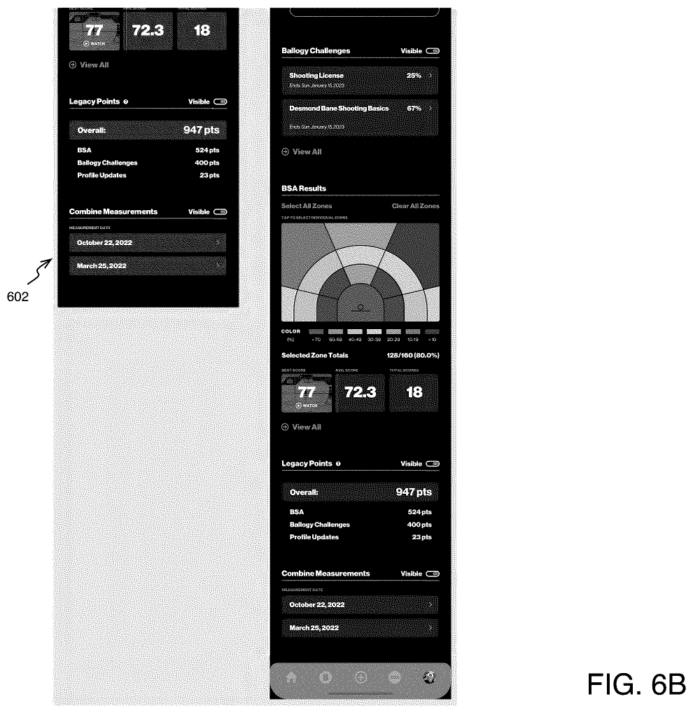

In implementations, recommendations may be suggested to a player and/or a coach, for example, via a “My Ballogy” screen portion of the app. See, for example, FIG. 6 and its associated subsections. A player may be provided, via the app's graphical user interface, an ability to “tap” on a particular zone of the basketball floor to prompt recommendations regarding workouts. For example, as may be seen at screen(s) 602 of FIGS. 6A-6B, for example, a particular zone of the basketball floor may appear “red” after a shooting assessment, indicating a larger number of missed shots for that zone. The player may wish to be assigned workouts designed to improve shot-making from that zone, so the player may select, as via a tap to the graphical user interface, the zone in question. The recommendation engine may then determine one or more appropriate workouts for the player, and the recommended workouts may be displayed to the player and/or an option to select one or more of the recommended workouts may be provided by the app. Similarly, coaches may be provided options to assign workouts based on a player's skills assessment, in some implementations.

Also, a player may elect to engage in a “free shoot” whereby the player simply takes shots from wherever they want on the basketball floor. Video analysis of the shooting session, such as via AI/machine-learning, may track makes and misses and may track where on the floor the shots occurred. Recommendations may be provided for this type of assessment as well, based at least in part on the analysis of hits and misses.

In some implementations, such as where video analysis may not be available and/or where AI/machine-learning is not available, recommendations may be made based at least in part on multiple results of skills assessment sessions. For example, if the aforementioned skills assessment is performed multiple times by a particular player and the results are recorded in the app (e.g., even if self-reported rather than via video analysis with or without AI/machine-learning), recommendations for workouts from the curriculum may be generated and communicated to the player, coach, parent, etc., as appropriate, by the app. Again, such recommendations may include communication between a user's electronic device (e.g., cell phone, tablet, notebook computer, etc.) and a remote computing device (e.g., server computing platform) by way of one or more communication techniques discussed herein.

In other implementations, a recommendation engine may design its own workout, such as a customized shooting workout, based on AI/machine-learning analysis of a player's shooting session (e.g., free shoot or structured routine, such as a skills assessment), rather than assigning a pre-existing workout from the app's catalog/curriculum.

As previously mentioned, coaches may design workouts and may select whether to make the workouts public or to keep them private (e.g., available to only specified individuals and/or groups). A recommendation engine may add such public-specified coach-designed workouts to the pool of available workouts that may be recommended.

Also, in embodiments, a coach or other Workout designer may be provided, via a sports social media application, an ability to not only indicate public or private status, but may also indicate one or more categories (e.g., shooting, dribbling, passing, etc.) that appropriately categorize their drill. See, for example, screen(s) 421 of FIG. 4C. A coach or other Workout designer may also be provided a graphical user interface element by which a coach or other Workout designer may indicate one or more particular zones of a shot chart, for example, that may correspond to the new Workout. For example, Workouts aimed at improving finishing around the rim might be categorized differently than Workouts directed at improving 3-point shots from the corner, and a coach or other Workout designer may indicate such via a graphical user interface element, such as a displayed shot chart, for example. This feature may also be implemented in connection with other aspects of a sports social media application, such as the skills assessment, for example. And, of course, such functionality is not limited to shooting drills. Dribbling, passing, and/or finishing drills, for example, may take place in various zones of a basketball court, and coaches or other Workout designers may indicate such zones via a sports social media application's graphical user interface, such as during the “create a Workout” process, such as indicated in FIG. 4 and its associated subsections. Further, a recommendation engine may consider such “zone” information when recommending particular skills, drills, Workouts, etc. to particular players and/or groups.

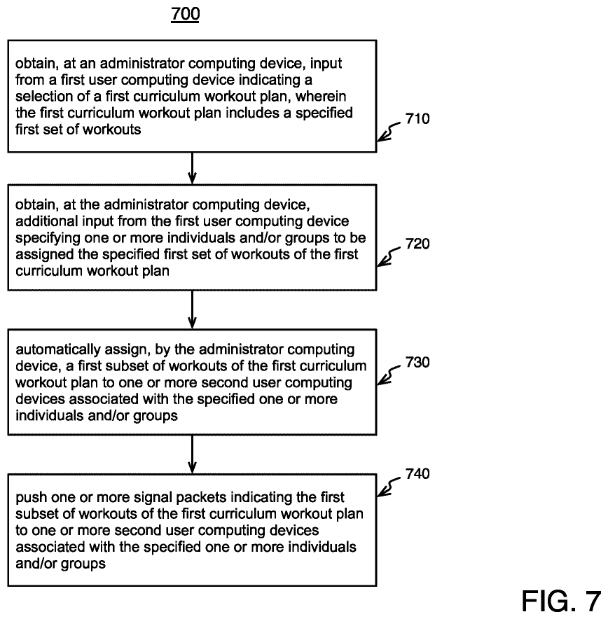

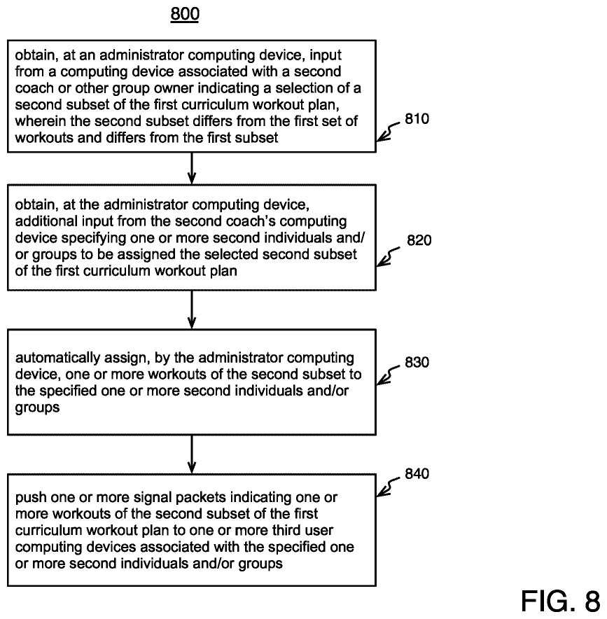

FIG. 7 and FIG. 8 are flow diagrams depicting embodiments 700 and 800 of example processes for athletic and/or skills assessment, development and/or performance, including automatically assigned curriculum workouts. Embodiments may include all of the operations, processes, techniques, approaches, etc. described, fewer than the operations, processes, techniques, approaches, etc. described, and/or more than the operations, processes, techniques, approaches, etc. described for example processes 700 and/or 800. Likewise, it should be noted that content acquired or produced, such as, for example, input signals, output signals, operations, results, etc. associated with the example provided may be represented via one or more analog and/or digital signals and/or signal packets. It should also be appreciated that even though one or more operations, processes, techniques, approaches, etc. are illustrated or described concurrently or with respect to a certain sequence, other sequences or concurrent operations processes, techniques, approaches, etc. may be employed. Further, it should be noted that operations, processes, techniques, approaches, etc. may be implemented, performed, etc. by any combination of hardware, firmware and/or software. In addition, although the description below references particular aspects and/or features illustrated in certain other figures, one or more operations, processes, techniques, approaches, etc. may be performed with other aspects and/or features.

It may be appreciated that one or more software and/or firmware agents implemented to facilitate example functionalities described in connection with example processes 700 and/or 800, including automatic assignment-type curriculum workout plans, for example, may be partitioned between administrator computing devices, such as servers 120, for example, and any number of remote and/or mobile devices, such as IoT-type devices 102, for example, that may be associated with one or more coaches, players, parents, etc.

For example process 700, as indicated at block 710, an administrator computing device may obtain input from a first user computing device indicating a selection of a first curriculum workout plan, wherein the first curriculum workout plan includes a specified first set of workouts, in one or more embodiments. Additionally, example process 700 may include the administrator computing device obtaining additional input from the first user computing device specifying one or more individuals and/or groups to be assigned the specified first set of workouts of the first curriculum workout plan, as indicated at block 720.

In one or more embodiments, as indicated at block 730, the administrator computing device may automatically assign a first subset of workouts of the first curriculum workout plan to one or more second user computing devices associated with the specified one or more individuals and/or groups, and one or more signal packets indicating the first subset of workouts of the first curriculum workout plan may be pushed to one or more second user computing devices associated with the specified one or more individuals and/or groups, as indicated at block 740, for example.

In one or more embodiments, pushing the one or more signal packets indicating the first subset of workouts of the first curriculum workout plan to the one or more second user computing devices affects a transformation of a graphical user interface of a software agent executed at the one or more second user computing devices to notify the specified one or more individuals and/or groups of the assignment of the first subset of workouts of the first curriculum workout plan, for example. Also, in one or more embodiments, the first user computing device comprises a group owner computing device. Additionally, the first curriculum workout plan is directed to developing one or more skills associated with one or more sports for the specified one or more individuals and/or groups, for example.

In one or more embodiments, the first curriculum workout plan is directed to developing one or more skills associated with a sport of basketball, and wherein the specified one or more individuals and/or groups comprise one or more basketball players. Further, for example, the specified first set of workouts of the curriculum workout plan includes one or more workouts selected from one or more of a plurality of skills categories including basketball shooting, passing, dribbling, finishing, conditioning, rebounding defense, and/or mindset, in one or more embodiments.

Example process 700 may further comprise displaying, to the specified one or more individuals and/or groups via graphical user interfaces of copies of a sports social media application executed at the respective second user computing devices, instructions and/or guidance regarding how and/or when to perform individual workouts of the specified first set of workouts. Also, example process 700 may additionally include obtaining, by the administrator computing device, signals and/or states representative of results and/or scores for one or more individual workouts of the specified first set of workouts from one or more of the respective second user computing devices.

In one or more embodiments, the specified first set of workouts is to be assigned to the specified one or more individuals and/or groups during a first season (e.g., period of time) of a cyclical sporting calendar. Further, the first season comprises a preseason, and the cyclical sporting calendar may comprise an offseason, the preseason, and/or an in-season, for example. In one or more embodiments, the first curriculum workout plan specifies different sets of workouts for the preseason, the in-season, and/or the offseason. Further, the different set of workouts for the preseason, the in-season, and/or the offseason are automatically assigned to the specified one or more individuals and/or groups at times and/or dates specified for a given season, in one or more embodiments. In one or more embodiments, the group owner computing device is associated with a coach, and the different set of workouts for the preseason, the in-season, and/or the offseason may be automatically assigned to the specified one or more individuals and/or groups without further intervention by the coach. Example process 700 may also include obtaining, at the administrator computing device, further input from the coach via the group owner computing device indicating a different subset of workouts to assign to the specified one or more individuals and/or groups for a particular season of the preseason, in-season, and/or offseason, in one or more embodiments.

Turning now to FIG. 8, example process 800 may include an administrator computing device obtaining input from computing device associated with a second coach or other group owner indicating a selection of a second subset of a first curriculum workout plan, wherein second subset differs from the first set of workouts and differs from the first subset of the first curriculum workout plan, in one or more embodiments, as indicated at block 810. Additionally, example process 800 may include the administrator computing device obtaining additional input from the second coach's computing device specifying one or more second individuals and/or groups to be assigned the selected second subset of the first curriculum workout plan, as indicated at block 820.

Further, in one or more embodiments, as indicated at block 830, the administrator computing device may automatically assign one or more workouts of the second subset to the specified one or more second individuals and/or groups. Also, as indicated at block 840, one or more signal packets indicating the one or more workouts of the second subset of the first curriculum workout plan may be pushed to one or more third user computing devices associated with the specified one or more second individuals and/or groups, in one or more embodiments.

In one or more embodiments, different sets of workouts from the second subset are automatically assigned to the specified one or more second individuals and/or groups at times and/or dates specified for a given season of a specified sporting calendar. In embodiments, the specified sporting calendar comprises an offseason, a preseason, and an in-season, and/or wherein the different sets of workouts for the preseason, the in-season, and/or the offseason are automatically assigned to the specified one or more second individuals and/or groups without further intervention by the second coach, for example.

In one or more embodiments, the administrator computing device includes a database accessible, if given permission, to specified players and/or coaches or other group owners, wherein the database comprises a plurality of workouts, routines, assessment programs, drills, etc. for one or more sports and/or other athletic and/or skilled pursuits. The database may comprise one or more workouts, routines, assessment programs, and/or drills generated by application administrators and/or generated by coaches and/or players, for example. Further, for example, specified subsets of the workouts, routines, assessment programs, and/or drills of the database may be organized by the administrator computing device into one or more automatic assignment-type curriculum workout plans.

A need has been established to determine the shooting capabilities of a basketball player, for example. To evaluate a player's abilities, it may be advantageous to focus on the most frequently used shots in the modern game. A skills assessment (e.g., Ballogy Skills Assessment (BSA)), for example, may showcase a player's ability to make shots from close up, mid-range, and/or three-point distances, for example, to allow the player to display their ability from different spots on the floor. See, for example, FIG. 6 and its associated subsections. It may be advantageous for such an assessment to apply to all levels of players to give everyone a chance to see where and/or how they measure up. A number of variations of the tests have been utilized, and a 40-shot test may be advantageous because it may be accomplished in 4-minute increments, for example.

In embodiments, a skills assessment (e.g., BSA) may be advantageous because a disparity in training and evaluating talent in the amateur basketball space has been observed. A desire for skills assessment that could easily give a player, coach, or scout the ability to quickly tell what skill level a player is at has been identified. In embodiments, skills assessment (e.g., BSA-Ballogy Skills Assessment) may evaluate a player's shooting ability and/or may easily and/or readily compare the player's skill with their peers. There has been, historically, a missing “standardized” element to tracking and measuring skills development because, for example, everyone trains in their own way. For the player, the skills assessment gives them a chance to showcase their skills to the world.

It may be noted that a skills assessment (e.g., BSA) may be implemented, at least in part, via one or more computing devices, processing circuitry, sensors, communications technologies, etc. For example, skills assessment (e.g., BSA) may comprise, at least in part, a software application that may be executed by any of a wide range of electronic device types, such as those mentioned and/or discussed herein, including, to name but a few examples, IoT-type devices, tablet devices, cellular phones, notebook computers, desktop computers, etc.

In one or more embodiments, a skills assessment (e.g., BSA) may comprise the first objective skills metric that gives amateur athletes everywhere a chance to get noticed at the middle school, high school, and collegiate levels. Ballogy Skills Assessment is the first shooting test publicly accepted and endorsed by all levels of basketball by the NFHS, NHSBCA, NAIA, and NJCAA coaches alike, for example.

In embodiments, skills assessment (e.g., BSA) may be implemented in a sports social media application (e.g., software and/or firmware agent) that may be executed, at least in part, on a player's electronic device, such as IoT-type device 102, for example. Content related to the skills assessment (e.g., BSA) may be provided to the player's device by a software and/or firmware agent executed at an administrator computing device, such as server 120, for example. Collectively, software and/or firmware agents executed, at least in part, at one or more user devices (e.g., IoT-type devices associated with one or more players, coaches, parents, etc.) and/or at one or more administrator-type devices, such as servers 120, may be referred to herein as an “application” or “app.” Various functionalities and/or capabilities may be partitioned among the various devices, in some implementations. In other implementations, individual copies of a sports social media application and/or different versions of a sports social media application may be executed at different devices. For example, a first implementation of a sports social media application may be provided to mobile devices associated with players, coaches, etc., and/or a separate implementation may be executed at one or more servers, for example.

In implementations, if a user uploads their own profile, they receive a message stating that they'll receive their score and Legacy Points after their video is reviewed (e.g., by a human and/or via AI/machine-learning analysis). The video may be uploaded to Vimeo, for example, and then appears in our browser-based Admin Panel (and aspect of a sports social media application, such as may be executed at a computing device) where an administrator (e.g., staffer) can watch and manually denote makes and misses for each shot. In other implementations, analysis may be performed in an automated fashion via machine learning/AI. This process simultaneously time-stamps the video so that when it ultimately gets displayed in a sports social media application, viewers can tap on individual shots and automatically jump to that portion of the video. Once all shots are designated as make or miss, a score is produced and sent to the user, and the score/video appears in a sports social media application-wide BSA leaderboards.

If this video was recorded on behalf of someone, a (e.g., BSA) Score Code may be sent to either the email or phone number provided; whichever user profile syncs this code will have the resulting skills assessment (e.g., BSA) score/video associated with their profile. In implementations, the score/video may not appear on skills assessment (e.g., BSA) leaderboards until the code has been synced. If the skills assessment (e.g., BSA) video submission failed to meet all of the requirements, as judged by a staffer reviewing it and/or via AI/machine-learning analysis, it is deemed “invalid.” A notification is returned to the person who submitted the video letting them know that their submission was invalidated.

In implementations, video analysis may be performed by and/or assisted by artificial intelligence/machine learning aspects of a sports social media application.

In implementations, each user profile has a skills assessment (e.g., BSA) “credit” by default when they sign up for a sports social media application. Additional credits can be purchased. Each skills assessment (e.g., BSA) video submission uploaded uses one credit, regardless of whether the video is considered valid or not.

Before starting a skills assessment (e.g., BSA), the user can choose to record and submit for themselves (to add the resulting score to their own profile) or record on behalf of someone else. Choosing this second route may require either an email or phone number where a unique code will be sent after the skills assessment (e.g., BSA) recording is completed and uploaded. The user opens a skills assessment (e.g., BSA) session which triggers the computer vision (e.g., AI analysis, machine-learning, etc.). The user recording the video will see a real-time score update as each shot is attempted. Each shot is designated as either a make or a miss from the computer vision.

At the end of their 40 shots (or time expiration), for example, they should already see their score. It is up to them to decide whether or not they want to upload their video. If they do, all of the same mechanics apply as before, except that staff may review videos to modify or invalidate inaccuracies in Vision (e.g., AI/machine-learning analysis), but doesn't need to for scores to be populated on a sports social media application. Immediately after uploading, they should receive their Legacy Points (equivalent to their BSA score+100 additional points). If the score was for someone else, they should immediately receive their code to sync. That score will sync with their profile and remain there, in one or more embodiments.

In embodiments, a (e.g., Ballogy) Pre-Camp Assessment may be based on Dribbling, Layups/Finishing, and Shooting Fundamental Skills. In implementations, there is a goal range requirement for each skill. Dependent on the outcome of the given skill assessment will determine the score for each Fundamental Skill, for example.

In embodiments, (e.g., Ballogy) scores for each fundamental skill assessment may be between 1-3 with 1=Beginner, 2=Intermediate, 3-Advanced, for example. In one or more embodiments, after completion of the three assessments, an average of the three scores may be taken, thereby determining an overall (e.g., Ballogy) score and grouping of the athlete based on skill level, for example.

In implementations, an average score among the three categories may be a deciding factor of where to place the camper (e.g., player).

In embodiments, a (e.g., Ballogy) score may comprise a benchmark for beginner, intermediate, and advanced skill levels allowing coaches to evaluate their athletes before camp or tryout settings. It was created for various reasons, including customized learning experience. A pre-camp assessment tool allows camp organizers to gain valuable insights into each camper's current skill level, in implementations. This information enables camp staff to tailor the camp experience to meet the unique needs of every participant, ensuring that both beginners and advanced players receive instruction that is appropriate for their abilities.

Efficient Resource Allocation. In embodiments, coaches may be assigned to each skill level. By assessing the participants' skills in advance, camp organizers may better allocate resources. For instance, they can assign coaches or instructors with the right expertise to work with specific skill levels. This ensures that resources are utilized efficiently, leading to a more effective camp experience, for example. Enhanced Camper Engagement. Leveling the playing field. Campers who feel that their individual goals and skill levels are acknowledged are likelier to be engaged and motivated throughout the camp. In embodiments, a pre-camp assessment tool may foster a sense of ownership and/or personal investment in the camp experience. Streamlined Camp Planning: In embodiments, a tool may simplify camp planning by providing data on camper skill levels and grouping. It may be considered the standard assessment for camps due to its comprehensive nature, providing valuable insights into athletes' strengths, weaknesses, and areas for improvement, thus enhancing the camp experience. The assessment measures important aspects of the game such as dribbling, finishing, and shooting, in embodiments.

In embodiments, a sports social media application may focus on particular specified shots. For example, an X-Out Layup drill showcases the ability of an athlete to finish at the rim within a short amount of time. In implementations, a Hot-shot drill is used as a shooting assessment as it highlights the ability to make consistent shots at various areas on the court. In embodiments, a specified order of assessment drills may be designed to progressively challenge athletes. For example, a specified order may include starting with dribbling skills, moving on to finishing and gradually advancing to more challenging drills like hot shots that require movement around the court, shooting under pressure, accuracy, and consistency. The length of time of an assessment may be structured to cover a comprehensive range of basketball skills within a reasonable timeframe, ensuring thorough evaluation while optimizing efficiency. Essentially, it may be advantageous to not lose the attention the athlete and to therefore create an assessment that could be completed in under 5 minutes, in embodiments.

Embodiments may include a recommendation engine to provide separate functionalities for players and coaches for basketball and/or other sports (and, potentially, outside of sports), for example.

For example, a user may open a sports social media application and launch a “shoot-around” session and shoot from any location on the court at random. In embodiments, vision technology (e.g., AI/machine learning analysis implemented in a sports social media application) may provide real-time data on make, miss, and/or location via a sports social media application. After a session is stopped, the user may be provided a report that shows makes, misses, shooting percentages from each shot location and/or overall shooting percentage, for example.

In one or more embodiments, generative AI may suggest areas for improvement based on shooting percentages from quadrants on a shot chart. In embodiments, a player may ask an AI-based recommendation engine, via a sports social media application, questions on how to get better at a particular skill in a search engine format. For example, a user may query “How can I improve my left-hand layups?”

In embodiments, a Vision (e.g., AI/machine-learning analysis) Shoot-Around Feature may include: A sports social media application gives real-time data on: Make, Miss, Location. After the “session” is done, a sports social media application may provide the user a report on makes and misses, for example. A sharable shot chart with shooting percent from each location may be generated. In embodiments, a player can input their geo-location (gym, school, or park). In embodiments, video may be accessible via a player's profile on a sports social media application. In embodiments, may work indoors at half-court. In implementations, without Wi-Fi access, video may be saved to the player's phone.

Embodiments may also include a recommendation engine for coaches, for example. In an embodiment, a coach may have an ability to upload game tape to a desktop version of a sports social media application (e.g., Ballogy app). From a vision/computer aspect, an algorithm, implemented in a sports social media application at least in part, may analyze game tape and/or may suggest tasks and/or Workouts for a coach to assign to their team and/or individuals via the sports social media application (e.g., Ballogy mobile app). For example, if a team produces a large number of turnovers, a vision aspect of a sports social media application may communicate with an AI algorithm and/or may suggest to a coach to assign a variation of ball-handling and/or passing drills, in one or more embodiments.

In embodiments, a coach may have the ability in a group to “ask” a generative AI aspect to create or suggest a Workout for a team or individual(s). In embodiments, generative AI may source public workouts and/or tasks to create a unique Workout for a coach to assign to their team. In embodiments, a sports social media application may tag individual tasks and/or Workout that are created properly, for example.

Also, in embodiments, a coach may instruct an AI aspect to analyze a video of a recorded task and/or Workout and to generate feedback for a team or individual player(s), for example.

In the context of the present patent application, the term “connection,” the term “component” and/or similar terms are intended to be physical, but are not necessarily always tangible. Whether or not these terms refer to tangible subject matter, thus, may vary in a particular context of usage. As an example, a tangible connection and/or tangible connection path may be made, such as by a tangible, electrical connection, such as an electrically conductive path comprising metal or other conductor, that is able to conduct electrical current between two tangible components. Likewise, a tangible connection path may be at least partially affected and/or controlled, such that, as is typical, a tangible connection path may be open or closed, at times resulting from influence of one or more externally derived signals, such as external currents and/or voltages, such as for an electrical switch. Non-limiting illustrations of an electrical switch include a transistor, a diode, etc. However, a “connection” and/or “component,” in a particular context of usage, likewise, although physical, can also be non-tangible, such as a connection between a client and a server over a network, particularly a wireless network, which generally refers to the ability for the client and server to transmit, receive, and/or exchange communications, as discussed in more detail later.

In a particular context of usage, such as a particular context in which tangible components are being discussed, therefore, the terms “coupled” and “connected” are used in a manner so that the terms are not synonymous. Similar terms may also be used in a manner in which a similar intention is exhibited. Thus, “connected” is used to indicate that two or more tangible components and/or the like, for example, are tangibly in direct physical contact. Thus, using the previous example, two tangible components that are electrically connected are physically connected via a tangible electrical connection, as previously discussed. However, “coupled,” is used to mean that potentially two or more tangible components are tangibly in direct physical contact. Nonetheless, “coupled” is also used to mean that two or more tangible components and/or the like are not necessarily tangibly in direct physical contact, but are able to co-operate, liaise, and/or interact, such as, for example, by being “optically coupled.” Likewise, the term “coupled” is also understood to mean indirectly connected. It is further noted, in the context of the present patent application, since memory, such as a memory component and/or memory states, is intended to be non-transitory, the term physical, at least if used in relation to memory necessarily implies that such memory components and/or memory states, continuing with the example, are tangible.

Additionally, in the present patent application, in a particular context of usage, such as a situation in which tangible components (and/or similarly, tangible materials) are being discussed, a distinction exists between being “on” and being “over.” As an example, deposition of a substance “on” a substrate refers to a deposition involving direct physical and tangible contact without an intermediary, such as an intermediary substance, between the substance deposited and the substrate in this latter example; nonetheless, deposition “over” a substrate, while understood to potentially include deposition “on” a substrate (since being “on” may also accurately be described as being “over”), is understood to include a situation in which one or more intermediaries, such as one or more intermediary substances, are present between the substance deposited and the substrate so that the substance deposited is not necessarily in direct physical and tangible contact with the substrate.

A similar distinction is made in an appropriate particular context of usage, such as in which tangible materials and/or tangible components are discussed, between being “beneath” and being “under.” While “beneath,” in such a particular context of usage, is intended to necessarily imply physical and tangible contact (similar to “on,” as just described), “under” potentially includes a situation in which there is direct physical and tangible contact, but does not necessarily imply direct physical and tangible contact, such as if one or more intermediaries, such as one or more intermediary substances, are present. Thus, “on” is understood to mean “immediately over” and “beneath” is understood to mean “immediately under.”

It is likewise appreciated that terms such as “over” and “under” are understood in a similar manner as the terms “up,” “down,” “top,” “bottom,” and so on, previously mentioned. These terms may be used to facilitate discussion, but are not intended to necessarily restrict scope of claimed subject matter. For example, the term “over,” as an example, is not meant to suggest that claim scope is limited to only situations in which an embodiment is right side up, such as in comparison with the embodiment being upside down, for example. An example includes a flip chip, as one illustration, in which, for example, orientation at various times (e.g., during fabrication) may not necessarily correspond to orientation of a final product. Thus, if an object, as an example, is within applicable claim scope in a particular orientation, such as upside down, as one example, likewise, it is intended that the latter also be interpreted to be included within applicable claim scope in another orientation, such as right side up, again, as an example, and vice-versa, even if applicable literal claim language has the potential to be interpreted otherwise. Of course, again, as always has been the case in the specification of a patent application, particular context of description and/or usage provides helpful guidance regarding reasonable inferences to be drawn.

Unless otherwise indicated, in the context of the present patent application, the term “or” if used to associate a list, such as A, B, or C, is intended to mean A, B, and C, here used in the inclusive sense, as well as A, B, or C, here used in the exclusive sense. With this understanding, “and” is used in the inclusive sense and intended to mean A, B, and C; whereas “and/or” can be used in an abundance of caution to make clear that all of the foregoing meanings are intended, although such usage is not required. In addition, the term “one or more” and/or similar terms is used to describe any feature, structure, characteristic, and/or the like in the singular, “and/or” is also used to describe a plurality and/or some other combination of features, structures, characteristics, and/or the like. Likewise, the term “based on” and/or similar terms are understood as not necessarily intending to convey an exhaustive list of factors, but to allow for existence of additional factors not necessarily expressly described.

Furthermore, it is intended, for a situation that relates to implementation of claimed subject matter and is subject to testing, measurement, and/or specification regarding degree, that the particular situation be understood in the following manner. As an example, in a given situation, assume a value of a physical property is to be measured. If alternatively reasonable approaches to testing, measurement, and/or specification regarding degree, at least with respect to the property, continuing with the example, is reasonably likely to occur to one of ordinary skill, at least for implementation purposes, claimed subject matter is intended to cover those alternatively reasonable approaches unless otherwise expressly indicated. As an example, if a plot of measurements over a region is produced and implementation of claimed subject matter refers to employing a measurement of slope over the region, but a variety of reasonable and alternative techniques to estimate the slope over that region exist, claimed subject matter is intended to cover those reasonable alternative techniques unless otherwise expressly indicated.

To the extent claimed subject matter is related to one or more particular measurements, such as with regard to physical manifestations capable of being measured physically, such as, without limit, temperature, pressure, voltage, current, electromagnetic radiation, etc., it is believed that claimed subject matter does not fall within the abstract idea judicial exception to statutory subject matter. Rather, it is asserted, that physical measurements are not mental steps and, likewise, are not abstract ideas.

It is noted, nonetheless, that a typical measurement model employed is that one or more measurements may respectively comprise a sum of at least two components. Thus, for a given measurement, for example, one component may comprise a deterministic component, which in an ideal sense, may comprise a physical value (e.g., sought via one or more measurements), often in the form of one or more signals, signal samples and/or states, and one component may comprise a random component, which may have a variety of sources that may be challenging to quantify. At times, for example, lack of measurement precision may affect a given measurement. Thus, for claimed subject matter, a statistical or stochastic model may be used in addition to a deterministic model as an approach to identification and/or prediction regarding one or more measurement values that may relate to claimed subject matter.

For example, a relatively large number of measurements may be collected to better estimate a deterministic component. Likewise, if measurements vary, which may typically occur, it may be that some portion of a variance may be explained as a deterministic component, while some portion of a variance may be explained as a random component. Typically, it is desirable to have stochastic variance associated with measurements be relatively small, if feasible. That is, typically, it may be preferable to be able to account for a reasonable portion of measurement variation in a deterministic manner, rather than a stochastic matter as an aid to identification and/or predictability.

Along these lines, a variety of techniques have come into use so that one or more measurements may be processed to better estimate an underlying deterministic component, as well as to estimate potentially random components. These techniques, of course, may vary with details surrounding a given situation. Typically, however, more complex problems may involve use of more complex techniques. In this regard, as alluded to above, one or more measurements of physical manifestations may be modeled deterministically and/or stochastically. Employing a model permits collected measurements to potentially be identified and/or processed, and/or potentially permits estimation and/or prediction of an underlying deterministic component, for example, with respect to later measurements to be taken. A given estimate may not be a perfect estimate; however, in general, it is expected that on average one or more estimates may better reflect an underlying deterministic component, for example, if random components that may be included in one or more obtained measurements, are considered. Practically speaking, of course, it is desirable to be able to generate, such as through estimation approaches, a physically meaningful model of processes affecting measurements to be taken.

In some situations, however, as indicated, potential influences may be complex. Therefore, seeking to understand appropriate factors to consider may be particularly challenging. In such situations, it is, therefore, not unusual to employ heuristics with respect to generating one or more estimates. Heuristics refers to use of experience related approaches that may reflect realized processes and/or realized results, such as with respect to use of historical measurements, for example. Heuristics, for example, may be employed in situations where more analytical approaches may be overly complex and/or nearly intractable. Thus, regarding claimed subject matter, an innovative feature may include, in an example embodiment, heuristics that may be employed, for example, to estimate and/or predict one or more measurements.

It is further noted that the terms “type” and/or “like,” if used, such as with a feature, structure, characteristic, and/or the like, using “optical” or “electrical” as simple examples, means at least partially of and/or relating to the feature, structure, characteristic, and/or the like in such a way that presence of minor variations, even variations that might otherwise not be considered fully consistent with the feature, structure, characteristic, and/or the like, do not in general prevent the feature, structure, characteristic, and/or the like from being of a “type” and/or being “like,” (such as being an “optical-type” or being “optical-like,” for example) if the minor variations are sufficiently minor so that the feature, structure, characteristic, and/or the like would still be considered to be substantially present with such variations also present. Thus, continuing with this example, the terms optical-type and/or optical-like properties are necessarily intended to include optical properties. Likewise, the terms electrical-type and/or electrical-like properties, as another example, are necessarily intended to include electrical properties. It should be noted that the specification of the present patent application merely provides one or more illustrative examples and claimed subject matter is intended to not be limited to one or more illustrative examples; however, again, as has always been the case with respect to the specification of a patent application, particular context of description and/or usage provides helpful guidance regarding reasonable inferences to be drawn.

With advances in technology, it has become more typical to employ distributed computing and/or communication approaches in which portions of a process, such as signal processing of signal samples, for example, may be allocated among various devices, including one or more client devices and/or one or more server devices, via a computing and/or communications network, for example. A network may comprise two or more devices, such as network devices and/or computing devices, and/or may couple devices, such as network devices and/or computing devices, so that signal communications, such as in the form of signal packets and/or signal frames (e.g., comprising one or more signal samples), for example, may be exchanged, such as between a server device and/or a client device, as well as other types of devices, including between wired and/or wireless devices coupled via a wired and/or wireless network, for example.

An example of a distributed computing system comprises the so-called Hadoop distributed computing system, which employs a map-reduce type of architecture. In the context of the present patent application, the terms map-reduce architecture and/or similar terms are intended to refer to a distributed computing system implementation and/or embodiment for processing and/or for generating larger sets of signal samples employing map and/or reduce operations for a parallel, distributed process performed over a network of devices. A map operation and/or similar terms refer to processing of signals (e.g., signal samples) to generate one or more key-value pairs and to distribute the one or more pairs to one or more devices of the system (e.g., network). A reduce operation and/or similar terms refer to processing of signals (e.g., signal samples) via a summary operation (e.g., such as counting the number of students in a queue, yielding name frequencies, etc.). A system may employ such an architecture, such as by marshaling distributed server devices, executing various tasks in parallel, and/or managing communications, such as signal transfers, between various parts of the system (e.g., network), in an embodiment. As mentioned, one non-limiting, but well-known, example comprises the Hadoop distributed computing system. It refers to an open source implementation and/or embodiment of a map-reduce type architecture (available from the Apache Software Foundation, 1901 Munsey Drive, Forrest Hill, MD, 21050-2747), but may include other aspects, such as the Hadoop distributed file system (HDFS) (available from the Apache Software Foundation, 1901 Munsey Drive, Forrest Hill, MD, 21050-2747). In general, therefore, “Hadoop” and/or similar terms (e.g., “Hadoop-type,” etc.) refer to an implementation and/or embodiment of a scheduler for executing larger processing jobs using a map-reduce architecture over a distributed system. Furthermore, in the context of the present patent application, use of the term “Hadoop” is intended to include versions, presently known and/or to be later developed.

In the context of the present patent application, the term network device refers to any device capable of communicating via and/or as part of a network and may comprise a computing device. While network devices may be capable of communicating signals (e.g., signal packets and/or frames), such as via a wired and/or wireless network, they may also be capable of performing operations associated with a computing device, such as arithmetic and/or logic operations, processing and/or storing operations (e.g., storing signal samples), such as in memory as tangible, physical memory states, and/or may, for example, operate as a server device and/or a client device in various embodiments. Network devices capable of operating as a server device, a client device and/or otherwise, may include, as examples, dedicated rack-mounted servers, desktop computers, laptop computers, set top boxes, tablets, netbooks, smart phones, wearable devices, integrated devices combining two or more features of the foregoing devices, and/or the like, or any combination thereof. As mentioned, signal packets and/or frames, for example, may be exchanged, such as between a server device and/or a client device, as well as other types of devices, including between wired and/or wireless devices coupled via a wired and/or wireless network, for example, or any combination thereof. It is noted that the terms, server, server device, server computing device, server computing platform and/or similar terms are used interchangeably. Similarly, the terms client, client device, client computing device, client computing platform and/or similar terms are also used interchangeably. While in some instances, for ease of description, these terms may be used in the singular, such as by referring to a “client device” or a “server device,” the description is intended to encompass one or more client devices and/or one or more server devices, as appropriate. Along similar lines, references to a “database” are understood to mean, one or more databases and/or portions thereof, as appropriate.

It should be understood that for ease of description, a network device (also referred to as a networking device) may be embodied and/or described in terms of a computing device and vice-versa. However, it should further be understood that this description should in no way be construed so that claimed subject matter is limited to one embodiment, such as only a computing device and/or only a network device, but, instead, may be embodied as a variety of devices or combinations thereof, including, for example, one or more illustrative examples.

A network may also include now known, and/or to be later developed arrangements, derivatives, and/or improvements, including, for example, past, present and/or future mass storage, such as network attached storage (NAS), a storage area network (SAN), and/or other forms of device readable media, for example. A network may include a portion of the Internet, one or more local area networks (LANs), one or more wide area networks (WANs), wire-line type connections, wireless type connections, other connections, or any combination thereof. Thus, a network may be worldwide in scope and/or extent. Likewise, sub-networks, such as may employ differing architectures and/or may be substantially compliant and/or substantially compatible with differing protocols, such as network computing and/or communications protocols (e.g., network protocols), may interoperate within a larger network.

In the context of the present patent application, the term sub-network and/or similar terms, if used, for example, with respect to a network, refers to the network and/or a part thereof. Sub-networks may also comprise links, such as physical links, connecting and/or coupling nodes, so as to be capable to communicate signal packets and/or frames between devices of particular nodes, including via wired links, wireless links, or combinations thereof. Various types of devices, such as network devices and/or computing devices, may be made available so that device interoperability is enabled and/or, in at least some instances, may be transparent. In the context of the present patent application, the term “transparent,” if used with respect to devices of a network, refers to devices communicating via the network in which the devices are able to communicate via one or more intermediate devices, such as one or more intermediate nodes, but without the communicating devices necessarily specifying the one or more intermediate nodes and/or the one or more intermediate devices of the one or more intermediate nodes and/or, thus, may include within the network the devices communicating via the one or more intermediate nodes and/or the one or more intermediate devices of the one or more intermediate nodes, but may engage in signal communications as if such intermediate nodes and/or intermediate devices are not necessarily involved. For example, a router may provide a link and/or connection between otherwise separate and/or independent LANs.

In the context of the present patent application, a “private network” refers to a particular, limited set of devices, such as network devices and/or computing devices, able to communicate with other devices, such as network devices and/or computing devices, in the particular, limited set, such as via signal packet and/or signal frame communications, for example, without a need for re-routing and/or redirecting signal communications. A private network may comprise a stand-alone network; however, a private network may also comprise a subset of a larger network, such as, for example, without limitation, all or a portion of the Internet. Thus, for example, a private network “in the cloud” may refer to a private network that comprises a subset of the Internet. Although signal packet and/or frame communications (e.g. signal communications) may employ intermediate devices of intermediate nodes to exchange signal packets and/or signal frames, those intermediate devices may not necessarily be included in the private network by not being a source or designated destination for one or more signal packets and/or signal frames, for example. It is understood in the context of the present patent application that a private network may direct outgoing signal communications to devices not in the private network, but devices outside the private network may not necessarily be able to direct inbound signal communications to devices included in the private network.

The Internet refers to a decentralized global network of interoperable networks that comply with the Internet Protocol (IP). It is noted that there are several versions of the Internet Protocol. The term Internet Protocol, IP, and/or similar terms are intended to refer to any version, now known and/or to be later developed. The Internet includes local area networks (LANs), wide area networks (WANs), wireless networks, and/or long haul public networks that, for example, may allow signal packets and/or frames to be communicated between LANs. The term World Wide Web (WWW or Web) and/or similar terms may also be used, although it refers to a part of the Internet that complies with the Hypertext Transfer Protocol (HTTP). For example, network devices may engage in an HTTP session through an exchange of appropriately substantially compatible and/or substantially compliant signal packets and/or frames. It is noted that there are several versions of the Hypertext Transfer Protocol. The term Hypertext Transfer Protocol, HTTP, and/or similar terms are intended to refer to any version, now known and/or to be later developed. It is likewise noted that in various places in this document substitution of the term Internet with the term World Wide Web (“Web”) may be made without a significant departure in meaning and may, therefore, also be understood in that manner if the statement would remain correct with such a substitution.

Although claimed subject matter is not in particular limited in scope to the Internet and/or to the Web; nonetheless, the Internet and/or the Web may without limitation provide a useful example of an embodiment at least for purposes of illustration. As indicated, the Internet and/or the Web may comprise a worldwide system of interoperable networks, including interoperable devices within those networks. The Internet and/or Web has evolved to a public, self-sustaining facility accessible to potentially billions of people or more worldwide. Also, in an embodiment, and as mentioned above, the terms “WWW” and/or “Web” refer to a part of the Internet that complies with the Hypertext Transfer Protocol. The Internet and/or the Web, therefore, in the context of the present patent application, may comprise a service that organizes stored digital content, such as, for example, text, images, video, etc., through the use of hypermedia, for example. It is noted that a network, such as the Internet and/or Web, may be employed to store electronic files and/or electronic documents.

The term electronic file and/or the term electronic document are used throughout this document to refer to a set of stored memory states and/or a set of physical signals associated in a manner so as to thereby at least logically form a file (e.g., electronic) and/or an electronic document. That is, it is not meant to implicitly reference a particular syntax, format and/or approach used, for example, with respect to a set of associated memory states and/or a set of associated physical signals. If a particular type of file storage format and/or syntax, for example, is intended, it is referenced expressly. It is further noted an association of memory states, for example, may be in a logical sense and not necessarily in a tangible, physical sense. Thus, although signal and/or state components of a file and/or an electronic document, for example, are to be associated logically, storage thereof, for example, may reside in one or more different places in a tangible, physical memory, in an embodiment.

A Hyper Text Markup Language (“HTML”), for example, may be utilized to specify digital content and/or to specify a format thereof, such as in the form of an electronic file and/or an electronic document, such as a Web page, Web site, etc., for example. An Extensible Markup Language (“XML”) may also be utilized to specify digital content and/or to specify a format thereof, such as in the form of an electronic file and/or an electronic document, such as a Web page, Web site, etc., in an embodiment. Of course, HTML and/or XML are merely examples of “markup” languages, provided as non-limiting illustrations. Furthermore, HTML and/or XML are intended to refer to any version, now known and/or to be later developed, of these languages. Likewise, claimed subject matter are not intended to be limited to examples provided as illustrations, of course.

In the context of the present patent application, the term “Web site” and/or similar terms refer to Web pages that are associated electronically to form a particular collection thereof. Also, in the context of the present patent application, “Web page” and/or similar terms refer to an electronic file and/or an electronic document accessible via a network, including by specifying a uniform resource locator (URL) for accessibility via the Web, in an example embodiment. As alluded to above, in one or more embodiments, a Web page may comprise digital content coded (e.g., via computer instructions) using one or more languages, such as, for example, markup languages, including HTML and/or XML, although claimed subject matter is not limited in scope in this respect. Also, in one or more embodiments, application developers may write code (e.g., computer instructions) in the form of JavaScript (or other programming languages), for example, executable by a computing device to provide digital content to populate an electronic document and/or an electronic file in an appropriate format, such as for use in a particular application, for example. Use of the term “JavaScript” and/or similar terms intended to refer to one or more particular programming languages are intended to refer to any version of the one or more programming languages identified, now known and/or to be later developed. Thus, JavaScript is merely an example programming language. As was mentioned, claimed subject matter is not intended to be limited to examples and/or illustrations.

In the context of the present patent application, the terms “entry,” “electronic entry,” “document,” “electronic document,” “content,”, “digital content,” “item,” and/or similar terms are meant to refer to signals and/or states in a physical format, such as a digital signal and/or digital state format, e.g., that may be perceived by a user if displayed, played, tactilely generated, etc. and/or otherwise executed by a device, such as a digital device, including, for example, a computing device, but otherwise might not necessarily be readily perceivable by humans (e.g., if in a digital format). Likewise, in the context of the present patent application, digital content provided to a user in a form so that the user is able to readily perceive the underlying content itself (e.g., content presented in a form consumable by a human, such as hearing audio, feeling tactile sensations and/or seeing images, as examples) is referred to, with respect to the user, as “consuming” digital content, “consumption” of digital content, “consumable” digital content and/or similar terms. For one or more embodiments, an electronic document and/or an electronic file may comprise a Web page of code (e.g., computer instructions) in a markup language executed or to be executed by a computing and/or networking device, for example. In another embodiment, an electronic document and/or electronic file may comprise a portion and/or a region of a Web page. However, claimed subject matter is not intended to be limited in these respects.

Also, for one or more embodiments, an electronic document and/or electronic file may comprise a number of components. As previously indicated, in the context of the present patent application, a component is physical, but is not necessarily tangible. As an example, components with reference to an electronic document and/or electronic file, in one or more embodiments, may comprise text, for example, in the form of physical signals and/or physical states (e.g., capable of being physically displayed). Typically, memory states, for example, comprise tangible components, whereas physical signals are not necessarily tangible, although signals may become (e.g., be made) tangible, such as if appearing on a tangible display, for example, as is not uncommon. Also, for one or more embodiments, components with reference to an electronic document and/or electronic file may comprise a graphical object, such as, for example, an image, such as a digital image, and/or sub-objects, including attributes thereof, which, again, comprise physical signals and/or physical states (e.g., capable of being tangibly displayed). In an embodiment, digital content may comprise, for example, text, images, audio, video, and/or other types of electronic documents and/or electronic files, including portions thereof, for example.

Also, in the context of the present patent application, the term “parameters” (e.g., one or more parameters), “values” (e.g., one or more values), “symbols” (e.g., one or more symbols) “bits” (e.g., one or more bits), “elements” (e.g., one or more elements), “characters” (e.g., one or more characters), “numbers” (e.g., one or more numbers), “numerals” (e.g., one or more numerals) or “measurements” (e.g., one or more measurements) refer to material descriptive of a collection of signals, such as in one or more electronic documents and/or electronic files, and exist in the form of physical signals and/or physical states, such as memory states. For example, one or more parameters, values, symbols, bits, elements, characters, numbers, numerals or measurements, such as referring to one or more aspects of an electronic document and/or an electronic file comprising an image, may include, as examples, time of day at which an image was captured, latitude and longitude of an image capture device, such as a camera, for example, etc. In another example, one or more parameters, values, symbols, bits, elements, characters, numbers, numerals or measurements, relevant to digital content, such as digital content comprising a technical article, as an example, may include one or more authors, for example. Claimed subject matter is intended to embrace meaningful, descriptive parameters, values, symbols, bits, elements, characters, numbers, numerals or measurements in any format, so long as the one or more parameters, values, symbols, bits, elements, characters, numbers, numerals or measurements comprise physical signals and/or states, which may include, as parameter, value, symbol bits, elements, characters, numbers, numerals or measurements examples, collection name (e.g., electronic file and/or electronic document identifier name), technique of creation, purpose of creation, time and date of creation, logical path if stored, coding formats (e.g., type of computer instructions, such as a markup language) and/or standards and/or specifications used so as to be protocol compliant (e.g., meaning substantially compliant and/or substantially compatible) for one or more uses, and so forth.

Signal packet communications and/or signal frame communications, also referred to as signal packet transmissions and/or signal frame transmissions (or merely “signal packets” or “signal frames”), may be communicated between nodes of a network, where a node may comprise one or more network devices and/or one or more computing devices, for example. As an illustrative example, but without limitation, a node may comprise one or more sites employing a local network address, such as in a local network address space. Likewise, a device, such as a network device and/or a computing device, may be associated with that node. It is also noted that in the context of this patent application, the term “transmission” is intended as another term for a type of signal communication that may occur in any one of a variety of situations. Thus, it is not intended to imply a particular directionality of communication and/or a particular initiating end of a communication path for the “transmission” communication. For example, the mere use of the term in and of itself is not intended, in the context of the present patent application, to have particular implications with respect to the one or more signals being communicated, such as, for example, whether the signals are being communicated “to” a particular device, whether the signals are being communicated “from” a particular device, and/or regarding which end of a communication path may be initiating communication, such as, for example, in a “push type” of signal transfer or in a “pull type” of signal transfer. In the context of the present patent application, push and/or pull type signal transfers are distinguished by which end of a communications path initiates signal transfer.

Thus, a signal packet and/or frame may, as an example, be communicated via a communication channel and/or a communication path, such as comprising a portion of the Internet and/or the Web, from a site via an access node coupled to the Internet or vice-versa. Likewise, a signal packet and/or frame may be forwarded via network nodes to a target site coupled to a local network, for example. A signal packet and/or frame communicated via the Internet and/or the Web, for example, may be routed via a path, such as either being “pushed” or “pulled,” comprising one or more gateways, servers, etc. that may, for example, route a signal packet and/or frame, such as, for example, substantially in accordance with a target and/or destination address and availability of a network path of network nodes to the target and/or destination address. Although the Internet and/or the Web comprise a network of interoperable networks, not all of those interoperable networks are necessarily available and/or accessible to the public.

In the context of the particular patent application, a network protocol, such as for communicating between devices of a network, may be characterized, at least in part, substantially in accordance with a layered description, such as the so-called Open Systems Interconnection (OSI) seven layer type of approach and/or description. A network computing and/or communications protocol (also referred to as a network protocol) refers to a set of signaling conventions, such as for communication transmissions, for example, as may take place between and/or among devices in a network. In the context of the present patent application, the term “between” and/or similar terms are understood to include “among” if appropriate for the particular usage and vice-versa. Likewise, in the context of the present patent application, the terms “compatible with,” “comply with” and/or similar terms are understood to respectively include substantial compatibility and/or substantial compliance.

A network protocol, such as protocols characterized substantially in accordance with the aforementioned OSI description, has several layers. These layers are referred to as a network stack. Various types of communications (e.g., transmissions), such as network communications, may occur across various layers. A lowest level layer in a network stack, such as the so-called physical layer, may characterize how symbols (e.g., bits and/or bytes) are communicated as one or more signals (and/or signal samples) via a physical medium (e.g., twisted pair copper wire, coaxial cable, fiber optic cable, wireless air interface, combinations thereof, etc.). Progressing to higher-level layers in a network protocol stack, additional operations and/or features may be available via engaging in communications that are substantially compatible and/or substantially compliant with a particular network protocol at these higher-level layers. For example, higher-level layers of a network protocol may, for example, affect device permissions, user permissions, etc.

A network and/or sub-network, in an embodiment, may communicate via signal packets and/or signal frames, such as via participating digital devices and may be substantially compliant and/or substantially compatible with, but is not limited to, now known and/or to be developed, versions of any of the following network protocol stacks: ARCNET, AppleTalk, ATM, Bluetooth, DECnet, Ethernet, FDDI, Frame Relay, HIPPI, IEEE 1394, IEEE 802.11, IEEE-488, Internet Protocol Suite, IPX, Myrinet, OSI Protocol Suite, QsNet, RS-232, SPX, System Network Architecture, Token Ring, USB, and/or X.25. A network and/or sub-network may employ, for example, a version, now known and/or later to be developed, of the following: TCP/IP, UDP, DECnet, NetBEUI, IPX, AppleTalk and/or the like. Versions of the Internet Protocol (IP) may include IPv4, IPv6, and/or other later to be developed versions.

Regarding aspects related to a network, including a communications and/or computing network, a wireless network may couple devices, including client devices, with the network. A wireless network may employ stand-alone, ad-hoc networks, mesh networks, Wireless LAN (WLAN) networks, cellular networks, and/or the like. A wireless network may further include a system of terminals, gateways, routers, and/or the like coupled by wireless radio links, and/or the like, which may move freely, randomly and/or organize themselves arbitrarily, such that network topology may change, at times even rapidly. A wireless network may further employ a plurality of network access technologies, including a version of Long Term Evolution (LTE), WLAN, Wireless Router (WR) mesh, 2nd, 3rd, or 4th generation (2G, 3G, 4G, or 5G) cellular technology and/or the like, whether currently known and/or to be later developed. Network access technologies may enable wide area coverage for devices, such as computing devices and/or network devices, with varying degrees of mobility, for example.

A network may enable radio frequency and/or other wireless type communications via a wireless network access technology and/or air interface, such as Global System for Mobile communication (GSM), Universal Mobile Telecommunications System (UMTS), General Packet Radio Services (GPRS), Enhanced Data GSM Environment (EDGE), 3GPP Long Term Evolution (LTE), LTE Advanced, Wideband Code Division Multiple Access (WCDMA), Bluetooth, ultra-wideband (UWB), 802.11b/g/n, and/or the like. A wireless network may include virtually any type of now known and/or to be developed wireless communication mechanism and/or wireless communications protocol by which signals may be communicated between devices, between networks, within a network, and/or the like, including the foregoing, of course.

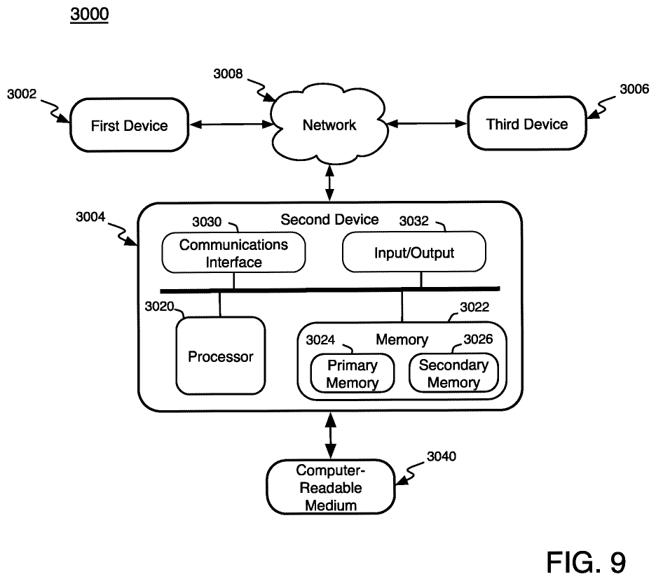

In one example embodiment, as shown in FIG. 9, a system embodiment may comprise a local network (e.g., device 3004 and medium 3040) and/or another type of network, such as a computing and/or communications network. For purposes of illustration, therefore, FIG. 9 shows an embodiment 3000 of a system that may be employed to implement either type or both types of networks. Network 3008 may comprise one or more network connections, links, processes, services, applications, and/or resources to facilitate and/or support communications, such as an exchange of communication signals, for example, between a computing device, such as 3002, and another computing device, such as 3006, which may, for example, comprise one or more client computing devices and/or one or more server computing device. By way of example, but not limitation, network 3008 may comprise wireless and/or wired communication links, telephone and/or telecommunications systems, Wi-Fi networks, Wi-MAX networks, the Internet, a local area network (LAN), a wide area network (WAN), or any combinations thereof.

Example devices in FIG. 9 may comprise features, for example, of a client computing device and/or a server computing device, in an embodiment. It is further noted that the term computing device, in general, whether employed as a client and/or as a server, or otherwise, refers at least to a processor and a memory connected by a communication bus. Likewise, in the context of the present patent application at least, this is understood to refer to sufficient structure within the meaning of 35 USC § 112 (f) so that it is specifically intended that 35 USC § 112 (f) not be implicated by use of the term “computing device” and/or similar terms; however, if it is determined, for some reason not immediately apparent, that the foregoing understanding cannot stand and that 35 USC § 112 (f), therefore, necessarily is implicated by the use of the term “computing device” and/or similar terms, then, it is intended, pursuant to that statutory section, that corresponding structure, material and/or acts for performing one or more functions be understood and be interpreted to be described at least in FIGS. 1-13 and in the text associated at least with the foregoing figure(s) of the present patent application.

Referring now to FIG. 9, in an embodiment, first and third devices 3002 and 3006 may be capable of rendering a graphical user interface (GUI) for a network device and/or a computing device, for example, so that a user-operator may engage in system use. Device 3004 may potentially serve a similar function in this illustration. Likewise, in FIG. 9, computing device 3002 (‘first device’ in figure) may interface with computing device 3004 (‘second device’ in figure), which may, for example, also comprise features of a client computing device and/or a server computing device, in an embodiment. Processor (e.g., processing device) 3020 and memory 3022, which may comprise primary memory 3024 and secondary memory 3026, may communicate by way of a communication bus 3015, for example. The term “computing device,” in the context of the present patent application, refers to a system and/or a device, such as a computing apparatus, that includes a capability to process (e.g., perform computations) and/or store digital content, such as electronic files, electronic documents, measurements, text, images, video, audio, sensor content, etc. in the form of signals and/or states. Thus, a computing device, in the context of the present patent application, may comprise hardware, software, firmware, or any combination thereof (other than software per se). Computing device 3004, as depicted in FIG. 9, is merely one example, and claimed subject matter is not limited in scope to this particular example.

For one or more embodiments, a device, such as a computing device and/or networking device, may comprise, for example, any of a wide range of digital electronic devices, including, but not limited to, desktop and/or notebook computers, high-definition televisions, game consoles, satellite television receivers, cellular telephones, tablet devices, wearable devices, personal digital assistants, mobile audio and/or video playback and/or recording devices, Internet of Things (IoT) type devices, endpoint and/or sensor nodes, gateway devices, streaming devices, or any combination of the foregoing. Further, unless specifically stated otherwise, a process as described, such as with reference to flow diagrams and/or otherwise, may also be executed and/or affected, in whole or in part, by a computing device and/or a network device. A device, such as a computing device and/or network device, may vary in terms of capabilities and/or features. Claimed subject matter is intended to cover a wide range of potential variations. For example, a device may include a numeric keypad and/or other display of limited functionality, such as a monochrome liquid crystal display (LCD) for displaying text, for example. In contrast, however, as another example, a web-enabled device may include a physical and/or a virtual keyboard, mass storage, one or more accelerometers, one or more gyroscopes, global positioning system (GPS) and/or other location-identifying type capability, and/or a display with a higher degree of functionality, such as a touch-sensitive color 2D or 3D display, for example.

As suggested previously, communications between a computing device and/or a network device and a wireless network may be in accordance with known and/or to be developed network protocols including, for example, global system for mobile communications (GSM), enhanced data rate for GSM evolution (EDGE), 802.11b/g/n/h, etc., and/or worldwide interoperability for microwave access (WiMAX). A computing device and/or a networking device may also have a subscriber identity module (SIM) card, which, for example, may comprise a detachable or embedded smart card that is able to store subscription content of a user, and/or is also able to store a contact list. It is noted, however, that a SIM card may also be electronic, meaning that is may simply be stored in a particular location in memory of the computing and/or networking device. A user may own the computing device and/or network device or may otherwise be a user, such as a primary user, for example. A device may be assigned an address by a wireless network operator, a wired network operator, and/or an Internet Service Provider (ISP). For example, an address may comprise a domestic or international telephone number, an Internet Protocol (IP) address, and/or one or more other identifiers. In other embodiments, a computing and/or communications network may be embodied as a wired network, wireless network, or any combinations thereof.

A computing and/or network device may include and/or may execute a variety of now known and/or to be developed operating systems, derivatives and/or versions thereof, including computing device operating systems, such as Windows, macOS, iOS, Linux, and/or the like, and/or mobile device operating systems, such as iOS, Android, Windows Mobile, and/or the like. A computing device and/or network device may include and/or may execute a variety of possible applications, such as a client software application enabling communication with other devices. For example, one or more messages (e.g., content) may be communicated, such as via one or more protocols, now known and/or later to be developed, suitable for communication of email, short message service (SMS), and/or multimedia message service (MMS), including via a network, such as a social network, formed at least in part by a portion of a computing and/or communications network, including, but not limited to, Facebook, Linkedin, Twitter, and/or Flickr, to provide only a few examples. A computing and/or network device may also include executable computer instructions to process and/or communicate digital content, such as, for example, textual content, digital multimedia content, sensor content, and/or the like. A computing and/or network device may also include executable computer instructions to perform a variety of possible tasks, such as browsing, searching, playing various forms of digital content, including locally stored and/or streamed video, and/or games such as, but not limited to, fantasy sports leagues. The foregoing is provided merely to illustrate that claimed subject matter is intended to include a wide range of possible features and/or capabilities.

In FIG. 9, computing device 3002 may provide one or more sources of executable computer instructions in the form physical states and/or signals (e.g., stored in memory states), for example. Computing device 3002 may communicate with computing device 3004 by way of a network connection, such as via network 3008, for example. As previously mentioned, a connection, while physical, may not necessarily be tangible. Although computing device 3004 of FIG. 9 shows various tangible, physical components, claimed subject matter is not limited to a computing devices having only these tangible components as other implementations and/or embodiments may include alternative arrangements that may comprise additional tangible components or fewer tangible components, for example, that function differently while achieving similar results. Rather, examples are provided merely as illustrations. It is not intended that claimed subject matter be limited in scope to illustrative examples.

Memory 3022 may comprise any non-transitory storage mechanism. Memory 3022 may comprise, for example, primary memory 3024 and secondary memory 3026, additional memory circuits, mechanisms, or combinations thereof may be used. Memory 3022 may comprise, for example, random access memory, read only memory, etc., such as in the form of one or more storage devices and/or systems, such as, for example, a disk drive including an optical disc drive, a tape drive, a solid-state memory drive, etc., just to name a few examples.

Memory 3022 may be utilized to store a program of executable computer instructions. For example, processor 3020 may fetch executable instructions from memory and proceed to execute the fetched instructions. Memory 3022 may also comprise a memory controller for accessing device readable-medium 3040 that may carry and/or make accessible digital content, which may include code, and/or instructions, for example, executable by processor 3020 and/or some other device, such as a controller, as one example, capable of executing computer instructions, for example. Under direction of processor 3020, a non-transitory memory, such as memory cells storing physical states (e.g., memory states), comprising, for example, a program of executable computer instructions, may be executed by processor 3020 and able to generate signals to be communicated via a network, for example, as previously described. Generated signals may also be stored in memory, also previously suggested.

Memory 3022 may store electronic files and/or electronic documents, such as relating to one or more users, and may also comprise a computer-readable medium that may carry and/or make accessible content, including code and/or instructions, for example, executable by processor 3020 and/or some other device, such as a controller, as one example, capable of executing computer instructions, for example. As previously mentioned, the term electronic file and/or the term electronic document are used throughout this document to refer to a set of stored memory states and/or a set of physical signals associated in a manner so as to thereby form an electronic file and/or an electronic document. That is, it is not meant to implicitly reference a particular syntax, format and/or approach used, for example, with respect to a set of associated memory states and/or a set of associated physical signals. It is further noted an association of memory states, for example, may be in a logical sense and not necessarily in a tangible, physical sense. Thus, although signal and/or state components of an electronic file and/or electronic document, are to be associated logically, storage thereof, for example, may reside in one or more different places in a tangible, physical memory, in an embodiment.

Algorithmic descriptions and/or symbolic representations are examples of techniques used by those of ordinary skill in the signal processing and/or related arts to convey the substance of their work to others skilled in the art. An algorithm is, in the context of the present patent application, and generally, is considered to be a self-consistent sequence of operations and/or similar signal processing leading to a desired result. In the context of the present patent application, operations and/or processing involve physical manipulation of physical quantities. Typically, although not necessarily, such quantities may take the form of electrical and/or magnetic signals and/or states capable of being stored, transferred, combined, compared, processed and/or otherwise manipulated, for example, as electronic signals and/or states making up components of various forms of digital content, such as signal measurements, text, images, video, audio, etc.

It has proven convenient at times, principally for reasons of common usage, to refer to such physical signals and/or physical states as bits, values, elements, parameters, symbols, characters, terms, numbers, numerals, measurements, content and/or the like. It should be understood, however, that all of these and/or similar terms are to be associated with appropriate physical quantities and are merely convenient labels. Unless specifically stated otherwise, as apparent from the preceding discussion, it is appreciated that throughout this specification discussions utilizing terms such as “processing,” “computing,” “calculating,” “determining”, “establishing”, “obtaining”, “identifying”, “selecting”, “generating”, and/or the like may refer to actions and/or processes of a specific apparatus, such as a special purpose computer and/or a similar special purpose computing and/or network device. In the context of this specification, therefore, a special purpose computer and/or a similar special purpose computing and/or network device is capable of processing, manipulating and/or transforming signals and/or states, typically in the form of physical electronic and/or magnetic quantities, within memories, registers, and/or other storage devices, processing devices, and/or display devices of the special purpose computer and/or similar special purpose computing and/or network device. In the context of this particular patent application, as mentioned, the term “specific apparatus” therefore includes a general purpose computing and/or network device, such as a general purpose computer, once it is programmed to perform particular functions, such as pursuant to program software instructions.

In some circumstances, operation of a memory device, such as a change in state from a binary one to a binary zero or vice-versa, for example, may comprise a transformation, such as a physical transformation. With particular types of memory devices, such a physical transformation may comprise a physical transformation of an article to a different state or thing. For example, but without limitation, for some types of memory devices, a change in state may involve an accumulation and/or storage of charge or a release of stored charge. Likewise, in other memory devices, a change of state may comprise a physical change, such as a transformation in magnetic orientation. Likewise, a physical change may comprise a transformation in molecular structure, such as from crystalline form to amorphous form or vice-versa. In still other memory devices, a change in physical state may involve quantum mechanical phenomena, such as, superposition, entanglement, and/or the like, which may involve quantum bits (qubits), for example. The foregoing is not intended to be an exhaustive list of all examples in which a change in state from a binary one to a binary zero or vice-versa in a memory device may comprise a transformation, such as a physical, but non-transitory, transformation. Rather, the foregoing is intended as illustrative examples.

Referring again to FIG. 9, processor 3020 may comprise one or more circuits, such as digital circuits, to perform at least a portion of a computing procedure and/or process. By way of example, but not limitation, processor 3020 may comprise one or more processors, such as controllers, microprocessors, microcontrollers, application specific integrated circuits, digital signal processors (DSPs), graphics processing units (GPUs), neural network processing units (NPUs), programmable logic devices, field programmable gate arrays, the like, or any combination thereof. In various implementations and/or embodiments, processor 3020 may perform signal processing, typically substantially in accordance with fetched executable computer instructions, such as to manipulate signals and/or states, to construct signals and/or states, etc., with signals and/or states generated in such a manner to be communicated and/or stored in memory, for example.

FIG. 9 also illustrates device 3004 as including a component 3032 operable with input/output devices, for example, so that signals and/or states may be appropriately communicated between devices, such as device 3004 and an input device and/or device 3004 and an output device. A user may make use of an input device, such as a computer mouse, stylus, track ball, keyboard, and/or any other similar device capable of receiving user actions and/or motions as input signals. Likewise, for a device having speech to text capability, a user may speak to a device to generate input signals. A user may make use of an output device, such as a display, a printer, etc., and/or any other device capable of providing signals and/or generating stimuli for a user, such as visual stimuli, audio stimuli and/or other similar stimuli.

According to an embodiment, a neural network may comprise a graph comprising nodes to model neurons in a brain. In this context, a “neural network” as referred to herein means an architecture of a processing device defined and/or represented by a graph including operators (represented by nodes in the graph) to model neurons that process input signals to generate output signals, and tensors (represented by edges in the graph) connecting the operators to represent input and/or output signal paths between and/or among operators (represented by nodes the graph). In particular implementations, a neural network may comprise a biological neural network, made up of real biological neurons, or an artificial neural network, made up of artificial neurons, for solving artificial intelligence (AI) problems, for example. In an implementation, such an artificial neural network may be implemented by one or more computing devices such as computing devices including a central processing unit (CPU), graphics processing unit (GPU), digital signal processing (DSP) unit and/or neural processing unit (NPU), just to provide a few examples. In a particular implementation, neural network weights and/or numerical coefficients associated with edges to represent input and/or output paths may reflect gains to be applied and/or whether an associated connection between connected nodes is to be excitatory (e.g., weight with a positive value) or inhibitory connections (e.g., weight with negative value). In an example implementation, a neuron may apply a neural network weight to input signals, and sum weighted input signals to generate a linear combination.

According to an embodiment, edges in a neural network connecting nodes may model synapses capable of transmitting signals (e.g., represented by real number values) between neurons. Responsive to receipt of such a signal, a node/neural may perform some computation to generate an output signal (e.g., to be provided to another node in the neural network connected by an edge). Such an output signal may be based, at least in part, on one or more weights and/or numerical coefficients associated with the node and/or edges providing the output signal. For example, such a weight may increase or decrease a strength of an output signal. In a particular implementation, such weights and/or numerical coefficients may be adjusted and/or updated as a machine learning process progresses. In an implementation, transmission of an output signal from a node in a neural network may be inhibited if a strength of the output signal does not exceed a threshold value.

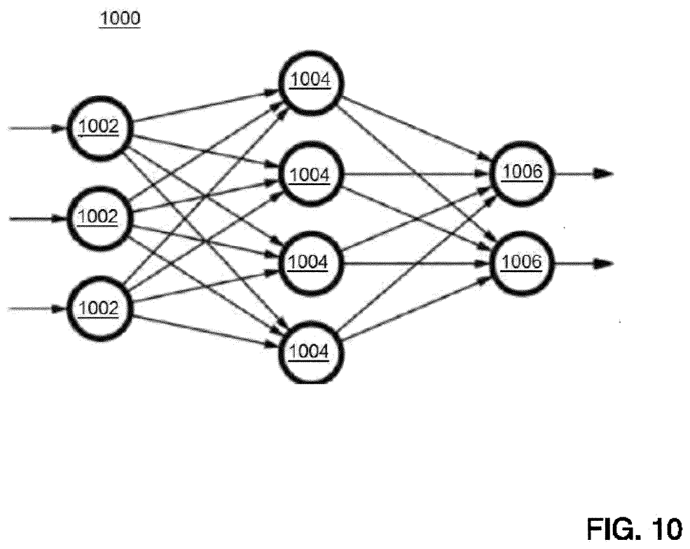

FIG. 10 is a schematic diagram of a neural network 1000 formed in “layers” in which an initial layer is formed by nodes 1002 and a final layer is formed by nodes 1006. Neural network (NN) 1000 may include an intermediate layer formed by nodes 1004. Edges shown between nodes 1002 and 1004 illustrate signal flow from an initial layer to an intermediate layer. Likewise, edges shown between nodes 1004 and 1006 illustrate signal flow from an intermediate layer to a final layer. While neural network 1000 shows a single intermediate layer formed by nodes 1004, it should be understood that other implementations of a neural network may include multiple intermediate layers formed between an initial layer and a final layer.

According to an embodiment, a node 1002, 1004 and/or 1006 may process input signals (e.g., received on one or more incoming edges) to provide output signals (e.g., on one or more outgoing edges) according to operators defined at associated nodes. At a node of neural network 1000, a set of one or more operations associated with the node may map one or more input signals to one or more output signals. In a particular implementation, such a set of one or more operations may be defined based, at least in part, on a weight associated with the node. One class of operators defined at nodes of neural network 1000 may comprise matrix multiplication-like operators such as, for example, convolutions, depth-wise convolutions, matrix multiplies, fully connected, batched matrix multiply and so forth, which may be further defined by associated weights. Another class of operators defined at nodes of neural network 1000 may comprise “activation functions” constructed as unary element-wise operators that introduce a non-linearity. Examples of such activation functions may include, for example, logistic (e.g., sigmoid and/or soft step), hyperbolic tangent, rectified linear unit, Gaussian error linear unit, Softplus, exponential linear unit, scaled exponential linear unit, leaky rectified linear unit, parametric rectified linear unit, sigmoid linear unit, Swish, Mish, Gaussian and/or growing cosine unit functions. Such activation functions may or may not be defined by trainable weights. A parametric rectified linear unit function, in particular, may be defined by a trainable alpha value. Additional element-wise operators defined at nodes of neural network 1000 may include multiply, add, subtract, negate, select and so forth. Nodes in neural network 1000 may also represent layout-like operators, such as Transpose, Reshape, Slice, Concat, Tile, just to provide a few examples.

Additionally, an “input value” as referred to herein means a value provided as an input parameter and/or signal to one or more operators defined and/or represented by a node in a neural network. Likewise, an “output value” as referred to herein means an output value provided by one or more operators defined and/or represented by a node of a neural network. In a particular implementation, an output value may be computed and/or generated according to one or more operators based on and/or responsive to one or more input values received at a node. In a particular implementation, an input value and/or output value may be structured, dimensioned and/or formatted as “tensors”. Thus, in this context, an “input tensor” as referred to herein means an expression of one or more input values according to a particular structure, dimension and/or format. Likewise in this context, an “output tensor” as referred to herein means an expression of one or more output values according to a particular structure, dimension and/or format.

According to an embodiment, neural network 1000 may be characterized as having a particular structure or topology based on, for example, a number of layers, number of nodes in each layers, operators implemented at each node, quantization of weights and quantization of input/output tensors. Neural network 1000 may be further characterized by weights to be assigned to nodes to affect operators at respective nodes. During execution, neural network 1000 may be characterized as having a particular state or “intermediate state” determined based on values/signals computed by nodes (e.g., as tensor values to be provided to nodes in a subsequent layer of nodes and/or an output tensor).

In particular implementations, neural networks may enable improved results in a wide range of tasks, including image recognition, speech recognition, just to provide a couple of example applications. To enable performing such tasks, features of a neural network (e.g., nodes, edges, weights, layers of nodes and edges) may be structured and/or configured to form “filters” that may have a measurable/numerical state such as a value of an output signal. Such a filter may comprise nodes and/or edges arranged in “paths” and are to be responsive to sensor observations provided as input signals. In an implementation, a state and/or output signal of such a filter may indicate and/or infer detection of a presence or absence of a feature in an input signal.

In particular implementations, intelligent computing devices to perform functions supported by neural networks may comprise a wide variety of stationary and/or mobile devices, such as, for example, automobile sensors, biochip transponders, heart monitoring implants, Internet of things (IoT) devices, kitchen appliances, locks or like fastening devices, solar panel arrays, home gateways, smart gauges, robots, financial trading platforms, smart telephones, cellular telephones, security cameras, wearable devices, thermostats, Global Positioning System (GPS) transceivers, personal digital assistants (PDAs), virtual assistants, laptop computers, personal entertainment systems, tablet personal computers (PCs), PCs, personal audio or video devices, personal navigation devices, just to provide a few examples.

According to an embodiment, a neural network may be structured in layers such that a node in a particular neural network layer may receive output signals from one or more nodes in an upstream layer in the neural network, and provide an output signal to one or more nodes in a downstream layer in the neural network. One specific class of layered neural networks may comprise a convolutional neural network (CNN) or space invariant artificial neural networks (SIANN) that enable deep learning. Such CNNs and/or SIANNs may be based, at least in part, on a shared-weight architecture of a convolution kernels that shift over input features and provide translation equivariant responses. Such CNNs and/or SIANNs may be applied to image and/or video recognition, recommender systems, image classification, image segmentation, medical image analysis, natural language processing (e.g., medical records processing), brain-computer interfaces, financial time series, just to provide a few examples.

Another class of layered neural network may comprise a recursive neural network (RNN) that is a class of neural networks in which connections between nodes form a directed cyclic graph along a temporal sequence. Such a temporal sequence may enable modeling of temporal dynamic behavior. In an implementation, an RNN may employ an internal state (e.g., memory) to process variable length sequences of inputs. This may be applied, for example, to tasks such as unsegmented, connected handwriting recognition or speech recognition, just to provide a few examples. In particular implementations, an RNN may emulate temporal behavior using finite impulse response (FIR) or infinite impulse response (IIR) structures. An RNN may include additional structures to control stored states of such FIR and IIR structures to be aged. Structures to control such stored states may include a network or graph that incorporates time delays and/or has feedback loops, such as in long short-term memory networks (LSTMs) and gated recurrent units.

According to an embodiment, output signals of one or more neural networks (e.g., taken individually or in combination) may at least in part, define a “predictor” to generate prediction values associated with some observable and/or measurable phenomenon and/or state. In an implementation, a neural network may be “trained” to provide a predictor that is capable of generating such prediction values based on input values (e.g., measurements and/or observations) optimized according to a loss function. For example, a training process may employ backpropagation techniques. “Backpropagation,” as referred to herein, is to mean a process of fitting parameters of a trained inference model such a model comprising one or more neural networks. In fitting parameters of a neural network, for example, backpropagation is to compute a gradient of a loss function with respect to the weights of the neural network. Based on such a computed gradient of a loss function, weights may be updated so as to minimize and/or reduce such a loss function. In one particular implementation, a gradient descent of a loss function, or variants such as stochastic gradient descent of a loss function, may be used. In training parameters of a neural network, backpropagation may comprise computing a gradient of a loss function with respect to individual weights by the chain rule, computing a gradient one layer at a time, iterating backward from the last layer to avoid redundant calculations of intermediate terms in the chain rule, for example. It should be understood, however, that this is merely an example of how a process of backpropagation may be applied, and claimed subject matter is not limited in this respect. In particular implementations, backpropagation may be used to iteratively update neural network weights to be associated with nodes and/or edges of a neural network based, at least in part on “training sets.” Such training sets may include training measurements and/or observations to be supplied as input values that are paired with “ground truth” observations. Based on a comparison of such ground truth observations and associated prediction values generated based on such input values in a training process, weights may be updated according to a loss function using backpropagation.

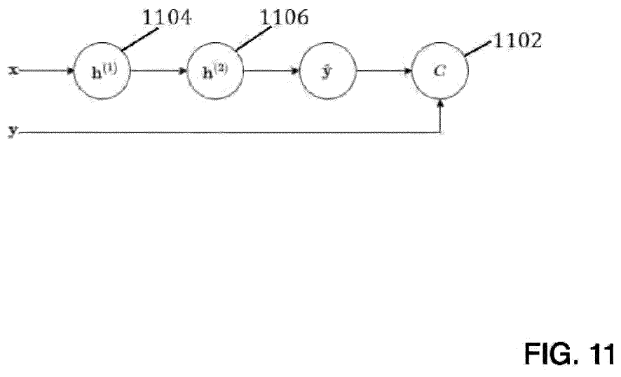

FIG. 11 is a flow diagram of an aspect of a training operation employing backpropagation to train parameters for a feedforward neural network, according to an embodiment. It should be understood, however, that this is merely an example of a type of neural network that may be trained using backpropagation, and that similar backpropagation techniques may be applied to train parameters of other types of neural networks without deviating from claimed subject matter. Training sets may be provided to such a training operation as pairs of vectors (x,y) where x is an input vector and y is a corresponding ground truth label. Input vector x may be provided as an input tensor to a first hidden layer 1104 to produce an output vector h⁽¹⁾, which is provided as an input to a second hidden layer 1106 to provide an output vector h⁽²⁾. An inference and/or prediction ŷ may be computed based, at least in part, on the output vector h⁽²⁾. A loss function C may be computed at 1102 based, at least in part, on inference and/or prediction y and ground truth label y.

In the particular embodiment of FIG. 11, inference and/or prediction y, and output vectors h⁽¹⁾ and h⁽²⁾ may be modelled as follows:

$$\begin{matrix}{h}^{( 1 )} = {g}^{( 1 )} ( {W}^{( 1 ) T} x + {b}^{( 1 )} ) \\ {h}^{( 2 )} = {g}^{( 2 )} ( {W}^{( 2 ) T} {h}^{( 1 )} + {b}^{( 2 )} ) \\ \hat{y} ( x ) = {W}^{( 3 ) T} {h}^{( 2 )} + {b}^{( 3 )} ,\end{matrix}$$

where:
   g⁽ⁱ⁾ is an activation function applied at nodes in hidden layer i;
   W⁽ⁱ⁾ is a matrix of weights such that weight

$${W}_{jk}^{( i )}$$

is to be applied at an edge going from node j in layer i−1 to node k in hidden layer i; and
   b⁽ⁱ⁾ is a bias matrix applied at hidden layer i.

In a particular implementation in which a feedforward neural network includes three or more hidden layers, computation of ŷ(x) may be generalized as follows:

$$\hat{y} ( x ) = {W}^{( N ) T} {h}^{( N - 1 )} + {b}^{( N )} .$$

Loss function C(y,ŷ) may be computed according to any one of several formulations of a loss function as described above. In a particular implementation, C(y,ŷ) may be differentiable such that <CWU

$$\frac{\partial C}{\partial {W}_{jk}^{( i )}}$$

may be determined using the chain rule and may be computed for any weight

$${W}_{j k}^{( i )} .$$

According to an embodiment, values for W⁽ⁱ⁾ may be determined iteratively for training sets (x,y) using a gradient descent technique.

In the preceding description, various aspects of claimed subject matter have been described. For purposes of explanation, specifics, such as amounts, systems and/or configurations, as examples, were set forth. In other instances, well-known features were omitted and/or simplified so as not to obscure claimed subject matter. While certain features have been illustrated and/or described herein, many modifications, substitutions, changes and/or equivalents will now occur to those skilled in the art. It is, therefore, to be understood that the appended claims are intended to cover all modifications and/or changes as fall within claimed subject matter.

What is claimed is:

1. A method, comprising:
obtaining, at an administrator computing device, input from a first user computing device indicating a selection of a first curriculum workout plan, wherein the first curriculum workout plan includes a specified first set of workouts;
obtaining, at the administrator computing device, additional input from the first user computing device specifying one or more individuals and/or groups to be assigned the specified first set of workouts of the first curriculum workout plan;
automatically assigning, by the administrator computing device, a first subset of workouts of the first curriculum workout plan to one or more second user computing devices associated with the specified one or more individuals and/or groups; and
pushing one or more signal packets indicating the first subset of workouts of the first curriculum workout plan to one or more second user computing devices associated with the specified one or more individuals and/or groups.

2. The method of claim 1, wherein pushing the one or more signal packets indicating the first subset of workouts of the first curriculum workout plan to the one or more second user computing devices affects a transformation of a graphical user interface of a software agent executed at the one or more second user computing devices to notify the specified one or more individuals and/or groups of the assignment of the first subset of workouts of the first curriculum workout plan.

3. The method of claim 1, wherein the first user computing device comprises a group owner computing device.

4. The method of claim 1, wherein the first curriculum workout plan is directed to developing one or more skills associated with a sport of basketball for the specified one or more individuals and/or groups, wherein the specified one or more individuals and/or groups comprise one or more basketball players.

5. The method of claim 4, wherein the specified first set of workouts of the curriculum workout plan includes one or more workouts selected from one or more of a plurality of skills categories including basketball shooting, passing, dribbling, finishing, conditioning, rebounding defense, and/or mindset.

6. The method of claim 1, further comprising:
displaying, to the specified one or more individuals and/or groups via graphical user interfaces of copies of an application executed at the respective second user computing devices, instructions and/or guidance regarding how and/or when to perform one or more individual workouts of the specified first set of workouts; and
obtaining, by the administrator computing device, signals and/or states representative of results and/or scores for the one or more individual workouts of the specified first set of workouts from one or more of the respective second user computing devices.

7. The method of claim 3, wherein the specified first set of workouts is to be automatically assigned to the specified one or more individuals and/or groups during a preseason of a cyclical sporting calendar, wherein the cyclical sporting calendar comprises an offseason, the preseason, and/or an in-season.

8. The method of claim 7, wherein the first curriculum workout plan specifies different sets of workouts for the preseason, the in-season, and/or the offseason.

9. The method of claim 8, wherein the different set of workouts for the preseason, the in-season, and/or the offseason are automatically assigned to the specified one or more individuals and/or groups at times and/or dates specified for a given season.

10. The method of claim 9, wherein the group owner computing device is associated with a coach, and wherein the different set of workouts for the preseason, the in-season, and/or the offseason are automatically assigned to the specified one or more individuals and/or groups without further intervention by the coach.

11. The method of claim 10, further comprising obtaining, at the administrator computing device, further input from the coach via the group owner computing device indicating a different subset of workouts to assign to the specified one or more individuals and/or groups for a particular season of the preseason, in-season, and/or offseason.

12. An apparatus, comprising: an administrator computing device comprising at least one processor coupled to at least one memory, wherein the at least one processor is to:
obtain, at the administrator computing device, input from a first user computing device indicating a selection of a first curriculum workout plan, wherein the first curriculum workout plan includes a specified first set of workouts;
obtain, at the administrator computing device, additional input from the first user computing device specifying one or more individuals and/or groups to be assigned the specified first set of workouts of the first curriculum workout plan;
automatically assign, by the administrator computing device, a first subset of workouts of the first curriculum workout plan to one or more second user computing devices associated with the specified one or more individuals and/or groups; and
push one or more signal packets indicating the first subset of workouts of the first curriculum workout plan from the administrator computing device to one or more second user computing devices associated with the specified one or more individuals and/or groups.

13. The apparatus of claim 12, wherein the one or more signal packets indicating the first subset of workouts of the first curriculum workout plan pushed to the one or more second user computing devices affects a transformation of a graphical user interface of a software agent executed at the one or more second user computing devices to notify the specified one or more individuals and/or groups of the assignment of the first subset of workouts of the first curriculum workout plan.

14. The apparatus of claim 12, wherein the first user computing device comprises a group owner computing device, and wherein the first curriculum workout plan is directed to developing one or more skills associated with a sport of basketball for the specified one or more individuals and/or groups, wherein the specified one or more individuals and/or groups comprise one or more basketball players, and

15. The apparatus of claim 14, wherein the specified first set of workouts of the curriculum workout plan includes one or more workouts selected from one or more of a plurality of skills categories including basketball shooting, passing, dribbling, finishing, conditioning, rebounding defense, and/or mindset.

16. The apparatus of claim 15, wherein the specified first set of workouts is to be automatically assigned to the specified one or more individuals and/or groups during a preseason of a cyclical sporting calendar, wherein the cyclical sporting calendar comprises an offseason, the preseason, and/or an in-season, and wherein the first curriculum workout plan specifies different sets of workouts for the preseason, the in-season, and/or the offseason.

17. The apparatus of claim 16, wherein the different set of workouts for the preseason, the in-season, and/or the offseason are automatically assigned to the specified one or more individuals and/or groups at times and/or dates specified for a given season.

18. An article, comprising: a non-transitory machine-readable medium having stored thereon instructions executable by at least one processor of an administrator computing device to:
obtain, at the administrator computing device, input from a first user computing device indicating a selection of a first curriculum workout plan, wherein the first curriculum workout plan includes a specified first set of workouts;
obtain, at the administrator computing device, additional input from the first user computing device specifying one or more individuals and/or groups to be assigned the specified first set of workouts of the first curriculum workout plan;
automatically assign, by the administrator computing device, a first subset of workouts of the first curriculum workout plan to one or more second user computing devices associated with the specified one or more individuals and/or groups; and
push one or more signal packets indicating the first subset of workouts of the first curriculum workout plan from the administrator computing device to one or more second user computing devices associated with the specified one or more individuals and/or groups.

19. The article of claim 18, wherein the first user computing device comprises a group owner computing device, and wherein the first curriculum workout plan is directed to developing one or more skills associated with a sport of basketball for the specified one or more individuals and/or groups, wherein the specified one or more individuals and/or groups comprise one or more basketball players, and wherein the specified first set of workouts of the curriculum workout plan includes one or more workouts selected from one or more of a plurality of skills categories including basketball shooting, passing, dribbling, finishing, conditioning, rebounding defense, and/or mindset.

20. The article of claim 19, wherein the specified first set of workouts is to be automatically assigned to the specified one or more individuals and/or groups during a preseason of a cyclical sporting calendar, wherein the cyclical sporting calendar comprises an offseason, the preseason, and/or an in-season, wherein the first curriculum workout plan specifies different sets of workouts for the preseason, the in-season, and/or the offseason, and wherein the different set of workouts for the preseason, the in-season, and/or the offseason are automatically assigned to the specified one or more individuals and/or groups at times and/or dates specified for a given season.
